(19) United States
(12) Patent Application Publication
(10) Pub. No.: US 2006/0121053 A1
(43) Pub. Date: Jun. 8, 2006

(54) High cell density process for growth of Listeria

(76) Inventors: Pamela Sweeney, Germantown, MD (US); Brian A. Russell, Germantown, MD (US)

Correspondence Address: JONES DAY, 222 EAST 41ST ST, NEW YORK, NY, 10017, US

(21) Appl. No.: 11/254,132

(22) Filed: Oct. 18, 2005

Related U.S. Application Data
(60) Provisional application No. 60/620,133, filed on Oct. 18, 2004.

(51) Int. Cl. A61K 39/02; C12N 1/20

(52) U.S. Cl. 424/200.1; 435/252.3

(57) ABSTRACT

The present invention relates to fed batch culture methods for high cell density growth of Listeria which produce cultures having an OD₆₀₀ greater than about 2.2 or higher. In particular, the invention provides methods for high cell density growth of Listeria comprising growth in a pH controlled bioreactor and, optionally, the gradual addition of a carbon source, e.g., glucose, with or without one or more additional nutrients, e.g., vitamins, when growth in the initial culture is nearly complete or complete. In one embodiment, the methods of the invention are used to produce Listeria-based compositions, e.g., vaccines comprising Listeria that express a tumor-associated antigen, e.g., an EphA2 antigenic peptide, for eliciting an immune response against hyperproliferative cells.

This application claims priority to U.S. Provisional Application No. 60/620,133, filed on Oct. 18, 2004, which is incorporated herein by reference in its entirety.

### 1. FIELD OF THE INVENTION

The invention relates to fed-batch methods for bioreactor production of high cell densities of Listeria. In particular, the invention provides methods for high cell density growth of Listeria, particularly fed-batch culturing Listeria cells in culture medium under conditions sufficient and for a time sufficient to achieve an OD₆₀₀ of greater than 2.2. In certain embodiments, fed-batch culturing comprises feeding with an additional carbon source after said Listeria culture reaches stationary phase. The invention further relates to high cell density cultures of Listeria produced by the methods of the invention. The Listeria may be used as whole cells in vaccines.

### 2. BACKGROUND OF THE INVENTION

Listeria monocytogenes (Listeria) is a Gram-positive facultative intracellular bacterium which has been studied for many years as a model for stimulating both innate and adaptive T cell-dependent antibacterial immunity. The ability of Listeria to effectively stimulate cellular immunity is based on its intracellular lifecycle. Upon infecting the host, the bacterium is rapidly taken up by phagocytes including macrophages and dendritic cells into a phagolysosomal compartment. The majority of the bacteria are subsequently degraded. Peptides resulting from proteolytic degradation of pathogens within phagosomes of infected APCs are loaded directly onto MHC class II molecules, and these MHC II-peptide complexes activate CD4+ “helper” T cells that stimulate the production of antibodies, and the processed antigens are expressed on the surface of the antigen presenting cell via the class II endosomal pathway. Within the acidic compartment, certain bacterial genes are activated including the cholesterol-dependent cytolysin, LLO, which can degrade the phagolysosome, releasing the bacterium into the cytosolic compartment of the host cell, where the surviving Listeria propagate. Efficient presentation of heterologous antigens via the MHC class I pathway requires de novo endogenous protein expression by Listeria. Within antigen presenting cells (APC), proteins synthesized and secreted by Listeria are sampled and degraded by the proteosome. The resulting peptides are shuttled into the endoplasmic reticulum by TAP proteins and loaded onto MHC class I molecules. The MHC I-peptide complex is delivered to the cell surface, which in combination with sufficient co-stimulation (signal 2) activates and stimulates cytotoxic T lymphocytes (CTLs) having the cognate T cell receptor to expand and subsequently recognize the MHC I-peptide complex.

Due to its ability to prime a potent CD4+/CD8+ T-cell mediated response via both MHC class I and class II antigen presentation pathways, Listeria is being developed for use in antigen-specific vaccines. Listeria-based vaccines and proteins expressed in Listeria are becoming more important as such products are becoming ready for the clinic or commercial use. Listeria-based vaccines have been studied for possible use against a wide variety of pathogens such as Mycobacterium tuberculosis (Miki et al., 2004, Infect Immun. 72:2014-21), human papillomavirus (Sewell et al., 2004, Arch Otolaryngol Head Neck Surg. 130:92-7), and human immunodeficiency virus (Lieberman et al., 2002, Vaccine 20:2007-10). Listeria-based vaccines have also been studied for the treatment and prevention of various cancers. A Listeria-based vaccine has been tested recently as a vaccine vector in a human clinical trial among normal healthy volunteers.

The use of Listeria-based vaccines on a large-scale would be limited, for example, because of the difficulty in obtaining sufficient quantities of Listeria. Currently available methods for the growth of Listeria yield low densities (less than about OD₆₀₀=2.2) and thus, result in a prolonged process, and one requiring inefficient use of raw materials, for production of Listeria in sufficient quantities for use therapeutically or prophylactically. The prolonged production process results in high manufacturing costs which in turn limits the access of many individuals to available therapy and results in shortages in supply of Listeria-based vaccines.

Large-scale production of bacteria generally involves fermenters or bioreactors. Currently available methods for the growth of bacteria include batch culture, continuous culture, and fed-batch methods. However, these methods have been developed for E. coli, yeasts, pseudomonads, and bacillus for use in preparing recombinant proteins. Up to recently, Listeria has not been a candidate for growth in large-scales because of the lack of a need. Thus, optimal conditions for large-scale growth of Listeria have not yet been determined. In addition, unlike E coli, Listeria cannot be grown using only inorganic nitrogen and requires an exogenous source of four vitamins (Welshimer, 1963, J. Bacteriol. 85:1156-1159) and at least one amino acid, cysteine (Tsai et al., 2003, Appl. Environ. Microbiol. 69:6943-6945). Listeria also do not possess the entire TCA cycle, but have a “split pathway” (Trivett et al., 1971, J. Bacteriol. 107:770-779) that may limit the flexibility of carbon utilization.

The recent discoveries of Listeria-based vaccines has generated interest in the large scale production of Listeria. Thus, a need exists for a cost-effective method for efficient high yield growth of Listeria. Such a method will reduce medical costs associated with therapies utilizing Listeria-based vaccines, improve supplies and, thus, make such therapies more widely available to the general public.

### 3. SUMMARY OF THE INVENTION

The invention relates to methods for producing high cell densities (e.g., OD₆₀₀ greater than about 2.2) of Listeria using fed-batch culture methods. The invention also relates to high cell density cultures of Listeria produced by fed-batch methods. In particular, the invention provides methods for high cell density growth of Listeria, particularly fed-batch culturing Listeria cells in culture medium under conditions sufficient and for a time sufficient to achieve an OD₆₀₀ of greater than 2.2. In certain embodiments, the fed-batch culturing comprises feeding with an additional carbon source after said Listeria culture reaches stationary phase. Additional parameters that can be used in the methods of the invention include the starting culture media and additional nutrients to be added with the additional carbon source, such as protein extracts, amino acids and vitamins. In one embodiment, Listeria cells are grown in a pH controlled bioreactor until the growth is complete or nearly complete, i.e., the culture enters the stationary phase, and then one or more additional nutrients are added gradually. The invention also provides particular fed-batch cell culture methods for high-yield production of a Listeria-based vaccine by recovering the Listeria from the cultures produced by methods of the invention.

The invention also relates to methods that increase the yield of Listeria-based vaccine production for, particularly, but not limited to, commercial scale production. The invention addresses difficulties in producing quantities of Listeria-based vaccines sufficient for clinical trials and therapeutic use. The invention also improves the cost, time and efficiency of large scale (e.g., greater than laboratory scale) production of vaccines.

In certain embodiments, the culture methods of the invention involve culturing Listeria cells in an appropriate cell culture medium until growth is complete or nearly complete and optionally, adding at least one additional nutrient in a gradual manner. The methods of the invention result in an OD₆₀₀ of the culture medium greater than 2.2, 2.5, 3.0, 3.5, 4.0, 4.5, 5.0, 5.5, 6.0, 6.5, 7.0, 7.5, 8.0, 9.0, 10.0, 15.0, 20.0, 25.0, 30.0, 35.0 or higher as measured, for example, after addition of the additional carbon source, e.g., 2, 3, 4, 5, 6, 8, 10, or 12 hours after addition of the additional carbon source. In other embodiments, the methods of the invention result in Listeria cultures that contain colony-forming units (cfu) per ml of 1.0×10⁸, 5.0×10⁸, 1.0×10⁹, 5.0×10⁹, 1.0×10¹⁰, 1.4×10¹⁰, 1.5×10¹⁰, 2.0×10¹⁰, 2.5×10¹⁰, or 2.8×10¹⁰ or higher. In general, the additional nutrient will be a carbon source, such as glucose, yeast extract or a combination of the two. Other nutrients, including, but not limited to, vitamin mixtures and amino acids, can be added. In order to prevent the accumulation of inhibitory organic acids, the additional carbon source is added gradually to the medium. It can be added at a constant rate, at an increasing rate (in a gradual, stepwise or linear fashion) or at an exponentially increasing rate. In a preferred embodiment, the additional carbon source is added at an exponentially increasing rate.

Any culture medium suitable for growth of Listeria can be used. In a preferred embodiment, the culture medium is tryptic soy medium or yeast growth medium. In certain embodiments, the culture medium does not contain a protein extract. In other embodiments, the culture medium is chemically defined.

The methods of the invention can be used for culturing of any Listeria strain, whether naturally occurring or recombinant. In a preferred embodiment, the Listeria strain is attenuated. In another preferred embodiment, the Listeria strain recombinantly expresses a heterologous peptide. In certain embodiments, the heterologous peptide is a tumor-associated antigen, such as EphA2. The heterologous peptide can also be a fusion protein, comprising a tumor-associated antigen, such as EphA2.

The methods of the invention result in significant improvement in yield of an Listeria-based vaccine (for example, at least 2-fold, 3-fold, 4-fold, 5-fold, 10-fold, 15-fold or 20-fold increase in yield) as compared to batch culture methods known in the art for culturing Listeria cells.

The invention provides high cell density cultures of Listeria having an OD₆₀₀ of the culture medium greater than 2.2, 2.5, 3.0, 3.5, 4.0, 4.5, 5.0, 5.5, 6.0, 6.5, 7.0, 7.5, 8.0, 9.0, 10.0, 15.0, 20.0, 25.0, 30.0, 35.0 or higher. In other embodiments, the invention provides Listeria cultures that contain colony-forming units (cfu) per ml of 1.0×10⁸, 5.0×10⁸, 1.0×10⁹, 5.0×10⁹, 1.0×10¹⁰, 1.4×10¹⁰, 1.5×10¹⁰, 2.0×10¹⁰, 2.5×10¹⁰, or 2.8×10¹⁰ or higher. The Listeria stain may be naturally occurring or recombinant. In a preferred embodiment, the Listeria strain is attenuated. In another preferred embodiment, the Listeria strain recombinantly expresses a heterologous peptide. In certain embodiments, the heterologous peptide is a tumor-associated antigen, such as EphA2. The heterologous peptide can also be a fusion protein, comprising a tumor-associated antigen, such as EphA2.

### 3.1 DEFINITIONS

As used herein, the term “bioreactor” means an apparatus used to carry out any kind of bioprocess; examples include a fermentor or enzyme reactor.

As used herein, the term “cell culture medium” means a medium suitable, but not necessarily sufficient, for culturing cells.

As used herein, the term “chemically defined media” or “chemically defined medium” means a cell culture medium prepared from purified ingredients, the exact composition of which is known.

As used herein, the term “fed-batch method”, means a culturing method wherein cells are cultured without removing media (except by evaporation and sampling), and thus, the total volume of media used remains essentially constant, or increased by addition of nutrient feeds, during the culturing method.

As used herein, the term “high density” means greater than OD₆₀₀ of 2.2, more preferably 2.5, 3.0, 3.5, 4.0, 4.5, 5.0, 5.5, 6.0, 6.5, 7.0, 7.5, 8.0, 9.0, 10.0, 15.0, 20.0, 25.0, 30.0, 35.0 or higher. In certain embodiments, the OD₆₀₀ is less than 11, less than 15, less than 20, less than 25, or less than 30. Cultures may contain colony-forming units (cfu) per ml of 1.0×10⁸, 5.0×10⁸, 1.0×10⁹, 5.0×10⁹, 1.0×10¹⁰, 1.4×10¹⁰, 1.5×10¹⁰, 2.0×10¹⁰, 2.5×10¹⁰, or 2.8×10¹⁰ or higher. In certain embodiments, the cfu/ml is less than 1.3×10⁹, 1.5×10⁹, 2.0×10⁹, 2.5×10⁹, 3.0×10⁹, 5.0×10⁹, 1.0×10¹⁰, 1.4×¹⁰, 1.5×10¹⁰, 2.0×10¹⁰, 5.0×10¹⁰, or 1.0×10¹¹.

As used herein, the term “Listeria-based vaccine” refers to a Listeria bacterium that has been engineered to express an antigenic peptide, or a composition comprising such a bacterium. A Listeria-based vaccine, when administered in an effective amount, elicits an immune response against the antigenic peptide. In a preferred embodiment, the Listeria is Listeria monocytogenes.

As used herein, the term “protein” includes peptides and polypeptides, and encompasses fusion proteins and fragments of proteins, polypeptides and antibodies (including domains such as extracellular domains, transmembrane domains, cytoplasmic domains, and immunoglobulin domains).

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

### 4. DETAILED DESCRIPTION OF THE INVENTION

The inventors have discovered that using the fed-batch methods disclosed herein high cell density cultures of Listeria can be obtained. Moreover, the yield and production of Listeria-based vaccines from cultured Listeria cells are greatly improved beyond those previously obtained by methods reported in the art.

The invention relates to fed-batch methods for producing high cell densities of Listeria by culturing in a pH controlled bioreactor and, optionally, supplementing the culture medium. In embodiments involving supplementation of the culture medium, after feeding is complete or nearly complete in the initial culture medium, feeding of one or more additional nutrients is initiated. This additional feeding allows the density of Listeria in the bioreactor to increase beyond that which can be achieved from the initial culture medium alone. Without being bound by any theory, it is believed that the gradual feeding of an additional nutrient prevents the cells from diverting excess carbon to formation of inhibitory organic acids, e.g., lactic acid. Optical densities (measured at 600 nm) may reach 15, 30, 39 or even higher (compared to OD₆₀₀˜2.0 obtained in previous reports). Cultures may contain colony-forming units (cfu) per ml of 1.0×10⁸, 5.0×10⁸, 1.0×10⁹, 5.0×10⁹, 1.0×10¹⁰, 1.4×10¹⁰, 1.5×10¹⁰, 2.0×10¹⁰, 2.5×10¹⁰, or 2.8×10¹⁰ or higher. In preferred embodiments, the optical densities and/or cfu/ml numbers achieved are obtained prior to any concentration step, e.g., centrifugation or filtration.

The methods of the invention involve fed-batch culture methods (both small and large scale, but preferably large scale (e.g., 100 L to 15000 L of culture medium used in the cell culture process)) involving the culture of Listeria cells. In a preferred embodiment, the Listeria cells express a heterologous protein. In a preferred embodiment, the Listeria cells can be used as a Listeria-based vaccine. Exemplary proteins, including those for use in Listeria-based vaccines, that can be produced by the methods of the invention are listed in section 4.3, below. Furthermore, uses of Listeria are described in section 4.6, below.

In a specific embodiment, the invention is directed to production-scale methods for producing Listeria, including Listeria-based vaccines, which methods achieve higher cell densities, as measured, for example, by OD₆₀₀, greater than conventional methods. These yields are improved over prior art methods in that (a) the amount of Listeria biomass per total volume of culture media is greater than that achieved in prior reported methods; and/or (b) the final concentration of Listeria-based vaccine is greater than that previously reported. Without being limited by any theory, the following factors individually and collectively contribute to the significant advantages of the methods of the invention: (1) bioreactor operating parameters such as temperature, pH, etc.; (2) nutrient feed additions; (3) feed timing; and (4) a combination thereof.

Fed-batch methods, generally, start with a volume of cell culture in an appropriate cell culture medium. Subsequently, feeds comprising one or more nutrients, e.g., a carbon source, such as glucose and/or a yeast extract, and, optionally, vitamins, amino acids, etc. (a “nutrient feed” may include any or all of these substances), are added periodically to the culture. Thus, in fed-batch methods, the culture volume increases solely due to the additions to the media (e.g., nutrient feeds).

Some nutrients inhibit cell growth at relatively low concentrations or may quickly create the buildup of inhibitory byproducts, e.g., lactic acid. Moreover, nutrient consumption can vary during the course of batch culture, due to expression of a recombinant protein, etc. The gradual feeding of additional nutrients prevents the cells from diverting excess carbon to formation of inhibitory organic acids. Thus, the gradual addition of nutrients during the course of the culture process, as provided by fed-batch culture methods enhances cell culture yields. In certain embodiments, the methods of the invention involve monitoring the levels of inhibitory byproducts, levels of one or more carbon sources, or levels of additional supplements.

The invention encompasses the culturing of Listeria cells for the production of a vaccine. In a preferred embodiment, Listeria monocytogenes is used. In a preferred embodiment, an attenuated strain of Listeria monocytogenes is used. In a preferred embodiment, a strain of Listeria monocytogenes recombinantly engineered to express an antigenic peptide is used.

The Listeria cells, preferably, are progeny of cells engineered through recombinant DNA techniques to express a vaccine, for example, a nucleic acid polymer encoding the vaccine is operably linked to a heterologous regulatory region that promotes high level expression of the protein. Techniques for recombinant production of vaccines are provided in section 4.2.3, below.

### 4.1 CULTURE METHODS OF THE INVENTION

The fed-batch methods described herein can be carried out in any vessel or container commonly used in the art for fed-batch culturing Listeria cells, such as test-tubes, flasks, bottles, or bioreactors, including, by way of example and not limitation, stirred-tank or airlift bioreactors (suspension reactors).

Suspension reactors allow for large scale mixing, direct sampling of cell mass, and precise monitoring and control of temperature, dissolved oxygen, and pH. In stirred tank reactors, cells are grown in stainless steel vessels which have height-to-diameter ratios of 1:1 to about 3:1. Mixing of the cell culture medium is accomplished using one or more agitators based on bladed-disk or marine-propeller patterns. Using this system, various methods of providing the cell culture with an adequate supply of oxygen have been developed. One of the more common methods is to sparge air or oxygen directly into the culture medium. Other methods of oxygen supply exist, including bubble-free aeration systems employing hollow fiber membrane aerators. For microbial cultures, suspension reactors are generally preferred.

In airlift reactors, a gas stream both mixes and aerates the cell culture. The height-to diameter-ratio of airlift reactors is generally 10:1 and is greater than that of stirred vessels. One of the advantages of airlift reactors is that they do not use motors or agitators; moreover, airlift reactors are relatively efficient in oxygen transfer and generate less shear stress on the culture than a stirred tank reactor.

If a bioreactor is not used, any device used in the art for maintaining culture conditions (such as temperature), e.g., an incubator, may be used.

The fed-batch culture methods described herein can be carried out using from 5 ml to 15000 L, or greater total volume of media used (including nutrient feeds). More specifically, for the large-scale production of Listeria, the methods of the invention can be carried out using volumes from 500 to 15000 L total volume of media used. The invention also contemplates laboratory-scale production, in a specific embodiment, where the total volume of medium used in the fed-batch process of the invention is less than 1 liter. The invention also contemplates scalability of the methods of the invention so that the methods of the invention can be carried out using between 1 L and 500 L total culture media (including nutrient feeds). In a particular embodiment of the invention, the fed-batch culture methods described herein can be carried out using 5 ml, 10 ml, 20 ml, 50 ml, 100 ml, 200 ml, 500 ml, 1 L, 2 L, 3, L, 4 L, 5L, 10 L, 100 L, 500 L, 1000 L, 5000 L, 10000 L or 15000 L total volume of media used (including nutrient feeds).

Any cell culture medium, suitable for growth of Listeria, known in the art may be suitable for use in the invention and can be determined using methods known in the art. In one embodiment, the culture medium is tryptic soy broth or yeast extract medium. Other suitable media include, but are not limited to, brain heart infusion, Listeria Fraser medium (Fraser et al., 1988, J. Food Prot. 51:762-765), etc.

In certain embodiments, the culture medium contains a protein extract. Preferably, when the medium comprises a protein extract, it comprises a yeast protein extract, and preferably, no other protein extract is used in the culture method. In other embodiments, the media comprise a plant protein extract, e.g., a wheat germ extract, rice extract, pea extract, cottonseed extract or soy extract; an animal protein extract, obtainable from such animals including, but not limited to, mammals, birds, fish, reptiles and amphibians; or an insect protein extract. Preferably, the protein extracts embodied herein are appropriate for the production of human therapeutic agents. Suitable yeast extract for the invention may be obtained from vendors (for example, Universal Flavors, Milan, Italy or Invitrogen Corp., Carlsbad, Calif.). Preferably, the yeast extract is supplied as a dried powder or as a sterile liquid and is produced through a manufacturing process that is free of animal-derived components. A preferred culture medium is Inoculum Expansion Medium which contains Yeastolate, ultrafiltered (25 g/L), Dextrose, anhydrous (10 g/L), KH₂PO₄ (9 g/L), and 10 N NaOH (5 mL/L). A more preferred culture medium is Inoculum Expansion Medium which contains Yeastolate, ultrafiltered (25 g/L), Dextrose, anhydrous (5 g/L), KH₂PO₄ (9 g/L), and 10 N NaOH (5 mL/L).

In an alternative embodiment, a chemically defined medium, free of any protein extract, is used. Chemically defined media are described in Friedman et al., 1961, J. Bacteriol. 82:528-537; Welshimer, 1963, J. Bacteriol. 85:1156-1159; Trivett et al., 1971, J. Bacteriol. 107:770-779 (D1O medium); Ralovich et al., 1977, Med. Microbiol. Immunol. 163:125-139; Siddiqi et al., 1989, Zentralblatt fur Bakteriologie 271:146-152; Premaratne et al., 1991, Applied Environ. Microbiol. 57:3046-3048 (MWB); Jones et al., 1995, J. Appl. Bacteriol. 78:66-70); and Tsai et al., 2003, Appl. Environ. Microbiol. 69:6943-6945 (HTM medium), all of which are herein incorporated by reference in their entireties. Optionally, additional amino acids or vitamin supplementation can be added depending on the particular Listeria strain used.

An initial starter culture is prepared by inoculating a small culture (e.g., 1, ml, 5 mls, 10 mls, 50 mls, 100 mls, 250 mls, or 500 mls) of Listeria using any suitable culture medium. Generally, it is desirable for an inoculum density of at least OD₆₀₀ of 1.0 to 2.0, or up to 4.0 or 5.0 at the time of inoculation.

The cell culture medium in the vessel or container for use in culturing is then inoculated, using any technique known in the art, with the Listeria cells suitable for use in the invention. Although the temperature, pH, agitation, aeration and inoculum density may vary, the following parameters are set forth by way of illustration and not limitation. The cell culture should be maintained at a temperature between 25 and 45° C. or 30 and 45° C.; in a preferred embodiment, the temperature is maintained between 36 and 39° C.; more preferably between 37° C. and 38° C., and, in a more preferred embodiment the temperature is maintained at 37° C. Furthermore, the pH of the culture medium should be monitored during the culture process so that the pH stays between 6.0 and 8.0; in a preferred embodiment of the invention, the pH should be maintained between 6.8 and 7.6; more preferably between 7.0 and 7.4. Generally, ammonium hydroxide, sodium hydroxide or sodium bicarbonate can be added to the culture medium to maintain a suitable pH, preferably a pH between 7.0 and 7.3. In a preferred embodiment, sodium hydroxide is added to the culture medium to maintain a suitable pH. Dissolved oxygen is maintained at a high concentration to ensure that the cells have the maximum capacity for growth and to avoid anaerobic metabolism, which often entails the production of possibly inhibitory organic acids. Sufficient aeration is provided to maintain a dissolved oxygen concentration of approximately 20% to 80%, preferably, approximately 40 to 60%, and, more preferably, approximately 50% air saturation in the culture.

The Listeria cells may be grown statically or with shaking. In a preferred embodiment, the cells are shaken. In a particular embodiment, impellor driven mixing is used for these culture methods. In a further embodiment, the rotational speed of the impellor is approximately 50-200 cm/sec tip speed, up to 500 cm/sec, preferably approximately 100 cm/sec tip speed, more preferably 200-300 cm/sec. In an alternative embodiment, airlift or other mixing/aeration systems may be used. In some embodiments, the culture is stationary.

The Listeria cells from the bioreactor, or other culture container, may be harvested from between a few hours to over 3 days from the inoculation of the bioreactor or other container, but preferably from 1 to 2 days from the inoculation of the medium.

### 4.1.1 Addition of Nutrients

During the culture of cells, nutrients are consumed in order to produce additional cells as well as any heterologous peptide. To address the depletion of nutrients as well as to induce high cell density growth of Listeria cells, in certain embodiments, one or more “nutrient feeds” are added to the bioreactor. In a preferred embodiment, an additional nutrient is added to the bioreactor when growth of the Listeria cells is complete or nearly complete, i.e., enters stationary phase. Preferably, the additional nutrient is a carbon source, e.g., glucose. Other suitable carbon sources include, but are not limited to, dextrose, fructose, glycerol, mannose, trehalose, cellobiose, maltose, glucosamine, N-acetylglucosamine, and N-acetylmuramic acid. The carbon source can be part of a complex nutrient source, e.g., the protein extracts as described above, containing a mix of carbohydrates, amino acids and vitamins. A preferred protein extract is a yeast extract. Combinations of carbon sources can also be used.

Because too high a concentration of some components may be toxic, nutrient feeds are generally added in doses, or added gradually, during the growth cycle. A gradual addition of a nutrient means that the nutrient is not added all at once. If a chemically defined medium is used, the reduced metabolic capacity of the cells may require slower addition of feed. The nutrient can be added in 2, 3, 4, 5, 6, 7, 8, 9, 10 or more separate doses. The nutrient can also be added continuously at flow rates of 0.5, 1.0, 2.0, 5.0, 10.0, 25.0 ml/hour or greater, or from about 0.5-200 ml/hour, 0.5-100 ml/hour, 1.0-50 ml/hour, 2.0-25 ml/hour.

When added continuously, the nutrient can be added at a constant rate, at a stepwise increasing rate, at a linearly increasing rate, or at an exponentially increasing rate. Preferably, the nutrient is added at an exponentially increasing rate to increase nutrient availability as the number of cells increase without feeding too much so that the excess is diverted into the production of inhibitory organic acids. Glucose concentration is maintained below 1.0 g/L to minimize these effects. In certain embodiments, the rate of increase can be calculated to give a particular doubling time.

A nutrient feed may optionally also contain amino acids and/or vitamins. Generally, the total nutrient feed volume will be 5 to 40%, 5 to 15% of the volume of the base culture medium. Preferably, the nutrient feed volume is 25 to 33% or below 10% of the volume of the base culture medium. Amino acids and vitamins are added in concentrated solutions. In a preferred embodiment, feeds are prepared using a 1000× vitamin solution containing 100 mg/L biotin, 1 g/L riboflavin, 1 g/L thiamine, and 1 mg/L thioctic acid. The 100× amino acid solution added contains 100 mg/L leucine, 77 mg/L cycsteine, and 200 mg/L each of: iso-leucine, valine, methionine, arginine, and histidine. Other suitable concentrated solutions can be determined by one of ordinary skill in the art.

Trace metals and minerals may also be added in quantities beyond those present in the batch medium. These may be added either as one-time supplements or additions to the feed. A preferred additive is magnesium sulfate.

Multiple nutrient feeds may be administered to cells to maintain an appropriate concentration of nutrients. For example, after inoculation of the cell culture, nutrient feeds may be administered to the cell culture once every few hours, once a day, once every 36 hours or once every 2 days. The culturing method of the invention may utilize between 1 and 9 nutrient feeds, or even more if necessary.

In certain embodiments, it is preferable to reduce the amount of glucose in the batch medium so that nutrient feeds are initiated earlier. In this way, the entire batch phase takes place in the presence of preferred nutrient feeds, e.g., a yeast-extract-supplemented feed, and faster growth is maintained.

### 4.1.2 Timing

In certain embodiments, the methods of the present invention involve the addition of one or more nutrients when growth in the initial culture medium is complete or nearly complete. Growth is considered to be complete or nearly complete when OD₆₀₀ and/or viable cell concentration (as measured by cfu/ml) ceases to increase or its increase slows, indicating a switch to a non-preferred nutrient.

Bacterial growth can be measured by standard methods known to one of skill in the art, including, but not limited to, optical density at 600 nm (OD₆₀₀). Bacterial growth can also be measured by determining nucleic acid ratios (Milner et al., 2001, Microbiol. 147:2689-2696).

A carbon source, such as glucose, can also be added when levels of the carbon source in the culture media drop below certain levels. For example, additional nutrient can be added when the glucose level drops below 5.0, 4.5, 4.0, 3.5, 3.0, 2.5 g/L or lower.

### 4.2 LISTERIA STRAINS

### 4.2.1 Wild Type Listeria Strains

Any wild-type strain of Listeria can be used in the methods of the present invention. In a preferred embodiment, the Listeria strain is Listeria monocytogenes.

### 4.2.2 Attenuated Listeria

While wild-type Listeria strains can be used in the methods of the invention, preferred Listeria strains, used for applications such as for administration to human subjects, are attenuated, for example, in their tissue tropism (e.g., in1B mutant) or ability to spread from cell to cell (e.g., actA mutant).

To allow the safe use of Listeria in treatment of humans and animals, the bacteria are preferably attenuated in their virulence for causing disease. The end result is to reduce the risk of toxic shock or other side effects due to administration of the Listeria to the patient. Such attenuated Listeria can be isolated by a number of techniques. Such methods include use of antibiotic-sensitive strains of microorganisms, mutagenesis of the microorganisms, selection for microorganism mutants that lack virulence factors, and construction of new strains of microorganisms with altered cell wall lipopolysaccharides.

In certain embodiments, the Listeria can be attenuated by the deletion or disruption of DNA sequences which encode for virulence factors which insure survival of the Listeria in the host cell, especially macrophages and neutrophils, by, for example, homologous recombination techniques and chemical or transposon mutagenesis. Many, but not all, of the studied virulence factors are associated with survival in macrophages such that these factors are specifically expressed within macrophages due to stress, for example, acidification, or are used to induce specific host cell responses, for example, macropinocytosis (Fields et al., 1986, Proc. Natl. Acad. Sci. USA 83:5189-5193).

As a method of insuring the attenuated phenotype and to avoid reversion to the non-attenuated phenotype, the Listeria may be engineered such that it is attenuated in more than one manner, e.g., a mutation in the pathway for lipid A production and one or more mutations to auxotrophy for one or more nutrients or metabolites, such as uracil biosynthesis, purine biosynthesis, and arginine biosynthesis.

In a preferred embodiment of the invention, the attenuated bacterium can cause less inflammatory reaction than the wild-type strain, e.g., at least 50%, preferably 70%, more preferably 90% less inflammation as measured in an infected mouse.

In a preferred embodiment, the attenuated bacterium is a mutant of Listeria monocytogenes which invades the host and is released into the cytosol of the infected cells with similar efficiencies as the wild-type strain, but it is not pathogenic, i.e., it doesn't cause a disease.

### 4.2.3 Recombinant Listeria -Constructs

One of ordinary skill in the art will recognize that a variety of plasmid construct backbones are available which are suitable for use in the assembly of a heterologous gene expression cassette. A particular plasmid construct backbone is selected based on whether expression of the heterologous gene from the bacterial chromosome or from an extra-chromosomal episome is desired. Construction of Listeria-based vaccines, including accessory sequences, is provided in detail in U.S. Provisional Patent Application Nos. 60/532,696, 60/602,588, 60/615,548, and 60/617,564, entitled “EphA2 Vaccines,” filed Dec. 24, 2003, Aug. 18, 2004, Oct. 1, 2004, and Oct. 7, 2004, respectively, U.S. Provisional Application Nos. 60/556,631, 60/615,470, and 60/617,544, entitled “Listeria-based EphA2 Vaccines,” filed Mar. 26, 2004, Oct. 1, 2004, and Oct. 7, 2004, and International Publication Nos. WO 2005/067460 and WO 2005/037233, each of which are herein incorporated by reference in their entireties.

The nucleotide sequences encoding a protein of interest may be obtained from any source of sequence information available to those of skill in the art (e.g., from Genbank, the literature, or by routine cloning). The DNA encoding the protein of interest can then be constructed by DNA amplification, molecular cloning or chemical synthesis. The nucleotide sequence coding for the protein can be inserted into an appropriate expression vector, i.e., a vector that contains the necessary elements for the transcription and translation of the inserted protein-coding sequence using methods which are well known to those skilled in the art. These methods include, for example, in vitro recombinant DNA techniques, synthetic techniques, and in vivo genetic recombination. See, for example, the techniques described in Sambrook et al., 2001, Molecular Cloning: A Laboratory Manual, 3^{rd} ed., Cold Spring Harbor Laboratory Press, and Ausubel et al., 2001, Current Protocols in Molecular Biology, John Wiley & Sons. Alternatively, RNA capable of encoding EphA2 antigenic polypeptide sequences may be chemically synthesized using, for example, synthesizers (see, e.g., the techniques described in Oligonucleotide Synthesis, 1984, Gait, M. J. ed., IRL Press, Oxford).

In a specific embodiment, the expression of a protein is regulated by a constitutive promoter. In another embodiment, the expression of a protein is regulated by an inducible promoter.

Expression vectors containing inserts of a gene encoding a peptide, polypeptide, protein or a fusion protein can be identified by three general approaches: (a) nucleic acid hybridization, (b) presence or absence of “marker” gene functions, and (c) expression of inserted sequences. In the first approach, the presence of a gene encoding a peptide, polypeptide, protein or a fusion protein in an expression vector can be detected by nucleic acid hybridization using probes comprising sequences that are homologous to an inserted gene encoding the peptide, polypeptide, protein or the fusion protein, respectively. In the second approach, the recombinant vector/host system can be identified and selected based upon the presence or absence of certain “marker” gene functions caused by the insertion of a nucleotide sequence encoding a polypeptide or a fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (e.g., fusion protein) expressed by the recombinant. Such assays can be based, for example, on the physical or functional properties of the fusion protein in in vitro assay systems.

Given as non-limiting examples, incorporation of the heterologous gene expression cassette into the bacterial chromosome of Listeria monocytogenes (Listeria) is accomplished with an integration vector that contains an expression cassette for a listeriophage integrase that catalyzes sequence-specific integration of the vector into the Listeria chromosome. For example, the integration vectors known as pPL1 and pPL2 program stable single-copy integration of a heterologous protein (e.g., EphA2-antigenic peptide) expression cassette within an innocuous region of the bacterial genome, and have been described in the literature (Lauer et al., 2002, J. Bacteriol. 184:4177-4178). The integration vectors are stable as plasmids in E. coli and are introduced via conjugation into the desired Listeria background. Each vector lacks a Listeria-specific origin of replication and encodes a phage integrase, such that the vectors are stable only upon integration into a chromosomal phage attachment site. Starting with a desired plasmid construct, the process of generating a recombinant Listeria strain expressing a desired protein(s) takes approximately one week. The pPL1 and pPL2 integration vectors are based, respectively, on the U153 and PSA listeriophages. The pPL1 vector integrates within the open reading frame of the comK gene, while pPL2 integrates within the tRNAArg gene in such a manner that the native sequence of the gene is restored upon successful integration, thus keeping its native expressed function intact. The pPL1 and pPL2 integration vectors contain a multiple cloning site sequence in order to facilitate construction of plasmids containing the heterologous protein expression cassette. Alternatively, incorporation of an antigenic peptide expression cassette into the Listeria chromosome can be accomplished through alleleic exchange methods, known to those skilled in the art. In particular, compositions in which it is desired to not incorporate a gene encoding an antibiotic resistance protein as part of the construct containing the heterologous gene expression cassette, methods of allelic exchange are desirable. For example, the pKSV7 vector (Camilli et al., 1993, Mol. Microbiol. 8:143-157), contains a temperature-sensitive Listeria Gram-positive replication origin which is exploited to select for recombinant clones at the non-permissive temperature that represent the pKSV7 plasmid recombined into the Listeria chromosome. The pKSV7 allelic exchange plasmid vector contains a multiple cloning site sequence in order to facilitate construction of plasmids containing the heterologous protein expression cassette, and also a chloramphenicol resistance gene. For insertion into the Listeria chromosome, the heterologous antigenic peptide expression cassette construct is optimally flanked by approximately 1 kb of chromosomal DNA sequence that corresponds to the precise location of desired integration. The pKSV7-heterologous protein expression cassette plasmid is introduced optimally into a desired bacterial strain by electroporation, according to standard methods for electroporation of Gram positive bacteria. Briefly, bacteria electroporated with the pKSV7-heterologous protein expression cassette plasmid are selected by plating on BHI agar media containing chloramphenicol (10 μg/ml), and incubated at the permissive temperature of 30° C. Single cross-over integration into the bacterial chromosome is selected by passaging several individual colonies for multiple generations at the non-permissive temperature of 41° C. in media containing chloramphenicol. Finally, plasmid excision and curing (double cross-over) is achieved by passaging several individual colonies for multiple generations at the permissive temperature of 30° C. in BHI media not containing chloramphenicol. Verification of integration of the heterologous protein (e.g., EphA2-antigenic peptide) expression cassette into the bacteria chromosome can be accomplished by PCR, utilizing a primer pair that amplifies a region defined from within the heterologous protein expression cassette to the bacterial chromosome targeting sequence not contained in the pKSV7 plasmid vector construct.

In other compositions, it may be desired to express the heterologous protein from a stable plasmid episome. Maintenance of the plasmid episome through passaging for multiple generations requires the co-expression of a protein that confers a selective advantage for the plasmid-containing bacterium. As non-limiting examples, the protein co-expressed from the plasmid in combination with the heterologous protein may be an antibiotic resistance protein, for example chloramphenicol, or may be a bacterial protein (that is expressed from the chromosome in wild-type bacteria), that can also confer a selective advantage. Non-limiting examples of bacterial proteins include enzyme required for purine or amino acid biosynthesis (selection under defined media lacking relevant amino acids or other necessary precursor macromolecules), or a transcription factor required for the expression of genes that confer a selective advantage in vitro or in vivo (Gunn et al., 2001, J. Immuol. 167:6471-6479). As a non-limiting example, pAM401 is a suitable plasmid for episomal expression of a selected heterologous protein in diverse Gram-positive bacterial genera (Wirth et al., 1986, J. Bacteriol 165:831-836).

### 4.3 PROTEINS TO BE EXPRESSED

It is understood that that methods of the present invention can be used for wild-type or attenuated strains that are not designed to recombinantly express a heterologous protein. For example, large quantities of Listeria can be made for commercial sale. Also, nonspecific antigen effects have demonstrated that Listeria has an adjuvant effect in slowing tumor growth (Pan et al., 1999, Cancer Res. 59:5264-5269).

In preferred embodiments of the invention, the methods of the present invention are use to produce heterologous proteins, including for use as Listeria-based vaccines.

As discussed above, in certain embodiments, the present invention relates to the use of Listeria that have been engineered to express an antigenic peptide. Without being bound by any mechanism, such Listeria are capable of eliciting an immune response to the antigenic peptide upon administration to a subject with a disease involving overexpression of the antigenic peptide, resulting in a cellular or humoral immune response against the endogenous antigen.

In principle, an antigenic peptide (sometimes referred to as an “antigenic polypeptide”) for use in the methods and compositions of the present invention can be any antigenic peptide that is capable of eliciting an immune response against antigen-expressing cells involved in a hyperproliferative disorder. Thus, an antigenic peptide can be a full-length polypeptide, or a fragment or derivative of an antigenic polypeptide that (1) displays antigenicity (ability to bind or compete with the antigen for binding to an anti-antigen antibody, (2) displays immunogenicity of the antigen (ability to generate antibody which binds to the antigen), or (3) contains one or more epitopes of the antigen.

In certain embodiments, the peptide corresponds to or comprises an antigen epitope that is exposed in a cancer cell but occluded in a non-cancer cell. In a preferred embodiment, the antigenic peptides preferentially include epitopes on the antigen that are selectively exposed or increased on cancer cells but not non-cancer cells.

The present invention further encompasses the use of a plurality of antigenic peptides in the compositions and methods of the present invention.

Fragments of antigen that are useful in the methods and compositions present invention may contain deletions, additions or substitutions of amino acid residues within the amino acid sequence. Preferably mutations result in a silent change, thus producing a functionally equivalent antigen.

The methods of the invention can be used for the production of, and the compositions of the invention can contain, a wide variety of proteins, e.g. including, but not limited to, soluble proteins, secreted proteins, transmembrane proteins, intracellular proteins, cytokines, cytokine receptors, transcription factors, signal transduction factors, DNA binding proteins, RNA binding proteins, kinases, toxins, and antibody secreted proteins.

The proteins produced using the fed-batch culture methods of the invention or contained in the compositions of the invention can be recovered and purified using the techniques disclosed herein or in the prior art.

The protein may be from or derived from any species of animals including, mammals such as non-primates and primates (e.g., humans) and infectious organisms (e.g., viruses, bacteria, parasites and fungi).

Fragments of proteins, polypeptides and antibodies (including domains such as extracellular domains, transmembrane domains, cytoplasmic domains, immunoglobulin domains) can also be produced by the methods of the invention or contained in the compositions of the invention.

The following sections provide lists, presented by way of example and not limitation, of proteins which can be produced by the invention or contained in the compositions of the invention:

### 4.3.1 Tumor-Associated Antigens

Tumor-associated antigens are reviewed in Berzofsky et al., 2004, J. Clin. Invest. 113:1515-1525 and Srinivasan et al., 2004, J. Transl. Med. 2:12-23, both of which are herein incorporated by reference in their entireties. Example of other tumor associated antigens include, but are not limited to, tyrosinase for melanoma, PSA and PSMA for prostate cancer and chromosomal cross-overs such as bcr/abl in lymphoma. However, many tumor associated antigens identified occur in multiple tumor types, and some, such as oncogenic proteins which actually cause the transformation event, occur in nearly all tumor types. For example, normal cellular proteins that control cell growth and differentiation, such as p53 and HER-2/neu, can accumulate mutations resulting in upregulation of expression of these gene products thereby making them oncogenic (McCartey et al. Cancer Research 1998 15:58 2601-5; Disis et al. Ciba Found. Symp. 1994 187:198-211). These mutant proteins can be the target of a tumor specific immune response in multiple types of cancer. Transforming proteins from oncogenic viruses such as E6 and E7 from HPV or EBNA1 from Epstein Barr virus (EBV) also occur in many tumor types and can be the target of a tumor specific immune response in multiple types of cancer (McKaig et al. Head Neck 1998 20(3):250-65; Punwaney et al. Head Neck 1999 21(1):21-9; Serth et al. Cancer Res. 1999 15:59(4):823-5; Pagano, J. S. Proc. Assoc. Am. Physicians 1999 111(6):573-80). Non-oncogenic host proteins such as MAGE and MUC family are also ubiquitous. Specifically, the MAGE family of antigens have been found in many different cancers including breast cancer, lung cancer, esophageal cancer, hepatic cancer, thyroid cancer, neuroblastoma, gastric cancer, multiple myeloma and melanoma (Gillespie, A. M. and Coleman, R. E. Cancer Treat. Rev. 1999 25(4):219-27). The MUC family of antigens has been associated with ovarian and endometrial cancer, breast cancer, multiple myeloma, pancreatic cancer, and colon and rectal cancer (Segal-Eiras, A. and Croce, M. V., 1997, Allergol. Immunopathol. 25(4): 176-81).

In a preferred embodiment, the tumor-associated antigen is EphA2. EphA2 is a 130 kDa receptor tyrosine kinase that is expressed in adult epithelia, where it is found at low levels and is enriched within sites of cell-cell adhesion (Zantek et al, 1999, Cell Growth & Differentiation 10:629; Lindberg et al., 1990, Molecular & Cellular Biology 10:6316). This subcellular localization is important because EphA2 binds ligands (known as EphrinsA1 to A5) that are anchored to the cell membrane (Eph Nomenclature Committee, 1997, Cell 90:403; Gale et al., 1997, Cell & Tissue Research 290: 227). The primary consequence of ligand binding is EphA2 autophosphorylation (Lindberg et al., 1990, supra). However, unlike other receptor tyrosine kinases, EphA2 retains enzymatic activity in the absence of ligand binding or phosphotyrosine content (Zantek et al., 1999, supra). EphA2 is upregulated on a large number hyperproliferating cells, including aggressive carcinoma cells.

In other embodiments, any of the other Eph receptors (EphA1, EphA3, EphA4, EphA5, EphA6, EphA7, EphA8, EphB1, EphB2, EphB3, EphB4, EphB5 and EphB6) or any of the ephrin ligands (EphrinA1, EphrinA2, EphrinA3, EphrinA4, EphrinA5, EphrinB 1, EphrinB2 and EphrinB3) which have been identified in mammals (see, e.g., Zhou et al.,1998, Pharmacol. Ther. 77:151-181) can be used.

### 4.3.2 Cytokines

Listeria have been shown to be capable of producing cytokines (Gossen et al., 1995, Biologicals, 23:135-43. Examples of cytokines include, but are not limited to, interleukin (“IL”)-1, IL-2, IL-3, IL-4, IL-5, IL-6, IL-7, IL-8, IL-9, IL-10, IL-11, IL-12, IL-13, IL-14, IL-15, IL-16, IL-17, IL-1 8, IL-19, IL-20, IL-21, IL-22, IL-23, interferon (“IFN”; e.g., IFN-α, IFN-β, and IFN-γ), tumor necrosis factor (“TNF”; e.g., TNF-α and TNF-β), nerve growth factor (“NGF”), platelet derived growth factor (“PDGF”), epidermal growth factor (“EGF”), tissue plasminogen activator (“TPA”; e.g., ACTIVASE® (alteplase) and TNKase™ (tenecteplase); Genentech), vascular endothelial growth factor (“VEGF”), granulocyte macrophage colony stimulating factor (“GM-CSF”), granulocyte colony stimulating factor (“G-CSF”; including NEUPOGEN® (filgrastim; Amgen) and the methionyl human G-CSF component of NEULASTA™ (pegfilgrastim; Amgen)), fibroblast growth factor (“FGF”; e.g., acid FGF and basic FGF), erythropoietin (“EPO”; e.g., EPOGEN® (epoeitin alfa) and ARANESP® (darbepoeitin alfa); Amgen), growth hormone (“GH”), growth hormone releasing hormone (“GHRH”), BNDF, connective tissue growth factor (“CTGF”), and corticotrophin releasing factor.

### 4.3.3 Viral Proteins

Examples of viral proteins useful for eliciting a reaction for a vaccine include, but are not limited to, influenza nucleoprotein (Pan et al., 1999, Cancer Res. 59:5264-5269), human papillomavirus nucleoprotein and lymphocytic choriomeningitis virus (LCMV) nucleoprotein, and HIV proteins, such as gag.

Other viral targets include respiratory syncytial virus (RSV), human papillomavirus (HPV), hepatitis C virus (HCV), Human metapneumovirus (hMPV), parainfluenza virus (PIV), Severe Acute Respiratory Syndrome (SARS).

### 4.3.4 Fusion Proteins

In certain embodiments of the present invention, a Listeria-based vaccine expresses an antigenic peptide that is a fusion protein. Thus, the present invention encompasses compositions and methods in which the antigenic peptides are fusion proteins comprising all or a fragment or derivative of an antigen operatively associated to a heterologous component, e.g., a heterologous peptide. Heterologous components can include, but are not limited to sequences which facilitate isolation and purification of the fusion protein. Heterologous components can also include sequences which confer stability to antigenic peptides. Such fusion partners are well known to those of skill in the art.

The present invention encompasses the use of fusion proteins comprising an antigenic polypeptide and a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). The fusion can be direct, but may occur through linker sequences. The heterologous polypeptide may be fused to the N-terminus or C-terminus of the antigenic polypeptide. Alternatively, the heterologous polypeptide may be flanked by antigenic polypeptide sequences. In preferred embodiments, the fusion protein comprises EphA2.

A fusion protein can comprise an antigenic polypeptide fused to a heterologous signal sequence at its N-terminus. Various signal sequences are commercially available. Prokaryotic heterologous signal sequences useful in the methods of the invention include, but are not limited to, the phoA secretory signal (Sambrook et al., eds., 1989, Molecular Cloning: A Laboratory Manual, 2nd ed., Cold Spring Harbor Laboratory, Cold Spring Harbor Laboratory Press, Cold Spring Harbor, N.Y.) and the protein A secretory signal (Pharmacia Biotech, Piscataway, N.J.).

The antigenic polypeptide can be fused to tag sequences, e.g., a hexa-histidine peptide, such as the tag provided in a pQE vector (QIAGEN, Inc., Chatsworth, Calif.), among others, many of which are commercially available for use in the methods of the invention. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced antigenic polypeptides, such as EphA2.

Any fusion protein may be readily purified by utilizing an antibody specific or selective for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

An affinity label can also be fused at its amino terminal to the carboxyl terminal of the antigenic polypeptide for use in the methods of the invention. The precise site at which the fusion is made in the carboxyl terminal is not critical. The optimal site can be determined by routine experimentation. An affinity label can also be fused at its carboxyl terminal to the amino terminal of the antigenic polypeptide for use in the methods and compositions of the invention.

A variety of affinity labels known in the art may be used, such as, but not limited to, the immunoglobulin constant regions (see also Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels are recognized by specific binding partners and thus facilitate isolation by affinity binding to the binding partner which can be immobilized onto a solid support. Some affinity labels may afford the EphA2 antigenic polypeptide novel structural properties, such as the ability to form multimers. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

Various leader sequences known in the art can be used for the efficient secretion of the antigenic polypeptide from bacterial cells such as Listeria (von Heijne, 1985, J. Mol. Biol. 184:99-105). Suitable leader sequences for targeting antigenic polypeptide expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (Maclntyre et al., 1990, Mol. Gen. Genet. 221:466-74; Kaiser et al., 1987, Science, 235:312-317).

In certain embodiments, the fusion partner comprises a non-antigenic polypeptide corresponding to an antigen associated with the cell type against which a therapeutic or prophylactic immune is desired. For example, with EphA2, the non-EphA2 polypeptide can comprise an epitope of a tumor-associated antigen, such as, but not limited to, MAGE-1, MAGE-2, MAGE-3, gp100, TRP-2, tyrosinase, MART-1, β-HCG, CEA, Ras, β-catenin, gp43, GAGE-1, BAGE-1, PSA, and MUC-1, 2, 3.

Polynucleotides encoding fusion proteins can be produced by standard recombinant DNA techniques. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. The expression of a fusion protein may be regulated by a constitutive, inducible or tissue-specific or -selective promoter. It will be understood by the skilled artisan that fusion proteins, which can facilitate solubility and/or expression, and can increase the in vivo half-life of the antigenic polypeptide and thus are useful in the methods of the invention. The antigenic polypeptides or peptide fragments thereof, or fusion proteins can be used in any assay that detects or measures specific antigenic polypeptides or in the calibration and standardization of such assay.

The methods of invention encompass the use of antigenic polypeptides or peptide fragments thereof, which may be produced by recombinant DNA technology using techniques well known in the art. Thus, methods for preparing the antigenic polypeptides of the invention by expressing nucleic acid containing antigenic gene sequences are described herein. Methods which are well known to those skilled in the art can be used to construct expression vectors containing, e.g., EphA2 antigenic polypeptide coding sequences (including but not limited to nucleic acids encoding all or an antigenic portion of a polypeptide) and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding EphA2 antigenic polypeptide sequences may be chemically synthesized using, for example, synthesizers (see, e.g., the techniques described in Oligonucleotide Synthesis, 1984, Gait, M. J. ed., IRL Press, Oxford).

In certain embodiments, the antigenic polypeptide is functionally coupled to an internalization signal peptide, also referred to as a “protein transduction domain,” that would allow its uptake into the cell nucleus. In certain specific embodiments, the internalization signal is that of Antennapedia (reviewed by Prochiantz, 1996, Curr. Opin. Neurobiol. 6:629 634, Hox A5 (Chatelin et al., 1996, Mech. Dev. 55:111 117), HIV TAT protein (Vives et al., 1997, J. Biol. Chem. 272:16010 16017) or VP22 (Phelan et al., 1998, Nat. Biotechnol. 16:440 443).

### 4.3.5 Other Proteins

Examples of cell surface receptors include, but are not limited to, CD2, CD3, CD4, CD5, CD6, CD7, CD8, CD9, CD10, CD11a, CD11c, CD14, CD17, CD19, C25, CD28, CD36, CD40, CD40 ligand, CD41, CD42, CD51, CD61, CD70, CD78, CTLA-4, ICAM (e.g., ICAM-1), integrin (e.g., integrin αᵥβ₃), bombesin receptor, complement geceptors (e.g., Clq complement receptor), chemokine receptors (e.g., chemokine (C-C) receptor and chemokine (C-X3-C) receptor 1 (“CX3CR1”)), cystic fibrosis transmembrane conductance regulator (“CFTR”), and cytokine receptors.

Cytokine receptors include, but are not limited to, IL-1 receptor (IL-1R), IL-2R, IL-3R, IL-4R, IL-5R, IL-6R, IL-7R, IL-8R, IL-9R, IL-10R, IL-11R, IL-12R, IL-13R, IL-14R, IL-15R, IL-16R, IL-17R, IL-18R, IL-19R, IL-20R, IL-21R, IL-22R, IL-23R, IFN-αreceptor, IFN-β receptor, IFN-γ receptor, TNF-α receptor, TNF-β receptor, NGF receptor, PDGF receptor, EGFR, TPA receptor, VEGFR, GM-CSF receptor, G-CSF receptor, FGF receptor, EPO receptor, GH receptor, and GHRH receptor. Proteins produced by the methods of the invention can also consist of sequences from more than one receptor. Such technology is exemplified by Traps as used by Regeneron, Inc., including an IL-4/IL-13 Trap.

Specific examples of other proteins include, but are not limited to, abl, acetyl CoA carboxylase beta, E-cadherin, gonadotropin, gonadotropin releasing hormone, acetylcholinesterase (“ACHE”), D-1 dopamine receptor (“DRD1”), effector cell protease receptor (“EPRI”), estrogen receptor, GABA receptor, glucagon receptor (“GCGR”), insulin receptor (“INSR”), alpha cardiac actin, acyl-CoA dehydrogenase (“ACADVL”), adiponectin (“ACRP30”), ADP-ribosylation factor-4, alpha-glucosidase, angiogenin, angiopoietin 1 (“ANG1”), angiopoietin 2 (“ANG2”), angiostatin, angiotensin 1-converting enzyme (“DCP1”), bactericidal/permeability-increasing protein (“BPI”), bc1-2, beta-catenin (“CTNNB 1”), beta-site APP-cleaving enzyme 2 (“BASE2”), bile salt export pump, BMP, brca1, brca2, c-fms, c-myc, calcitonin, calcium-binding protein in macrophages (“MRP14”), calsenilin (“DREAM/CSEN” or “CREAM” or “KCh IP3”), carnitine o-palmitoyltransferase (“CPT2”), catechol-o-methyltransferase (“COMT”), cathepsin K, CLCA homolog (“hCLCA2”), complement decay-accelerating factor (“DAF/CD55”), cyclin D1, cyclin E, cyclin T1, cyclin-dependent kinase inhibitor 1A (“p21” or “WAF1” or “CDKN1A” or “Cip1”), cyclin-dependent kinase inhibitor 2A (“CDKN2A”), cytochrome P-450, D-amino-acid oxidase (“DAO”), DNA binding protein (including “DDB 1”), DCC, desmoglein 1 (“DSG1”), dihydrofolate reductase (“DHFR”), disintegrin and metalloproteinase domain 33 (“ADAM 33”), recombinant DNase (e.g., PULMOZYME® (domase alfa; Genentech)), DNA methyltransferase (“DNMT3b”), DPP-IV, drebrin-1 dendritic spine protein (“DBN1”), endostatin, eotaxin (“CCL11”), factor IX, factor VIII, famesyl transferase, fibrillin (“FBN1”), FMS-related tyrosine kinase 1 (“FLTI”), forkhead box C2 (“FOXC2”), fos, galanin (“GAL”), gastric inhibitory polypeptide (“GIP”), glial cell line-derived neurotrophic factor (“GDNF”), glial growth factor (“GGF”), GGRP, ghrelin (“GHRL”), glucagon, glucagon-like peptide-1 (“GLP1”), glucokinase (“GCK”), glutamic acid decarboxylase 2, glutamic acid decarboxylase 3, glutamic acid decarboxylase, brain, membrane form, glycogen synthase kinase-3A (“GSK-3A”), glycogen synthase kinase-3B (“GSK-3B”), GRO2 oncogene or macrophage inflammatory protein-2-alpha precursor (“CXCL2”), gsp, H-ras, heat shock protein (“HSP”)-70, heparanase (“HPA”), hepatitis A virus cellular receptor (“HAVCR”), hepatitis B virus X interacting protein (“HBXIP”), hepsin (“HPN”), Her-2 (“ERBB2”), HGF, high mobility group box chromosomal protein 1 (“HMGB-1”), histone acetyltransferase (“HAT1”), histone deacetylase 1 (“HDAC1”), histone deacetylase 3 (“HDAC3”), HIV Tat Specific Factor 1 (“HTATSF1”), HMG CoA synthetase, HSP-90, 3-hydroxy-3-methylglutaryl-CoA reductase (“HMGCR”), hypoxia-inducible factor 1 (“HIF-1A”), hypoxia-inducible factor 1-alpha inhibitor (“HWF1AN”), iduronate 2-sulfatase (“IDS”), IGF-1, IGF-1R, IGF-2, IGF binding protein-2 (“IGFBP2”), IkB kinase (“IKBKB”), inositol polyphosphate phosphatase-like 1 (“SHIP-2”), insulin, interferon inducible protein (“CXCL10 (IP10)”), INI1/hSNF5, IL-1R antagonist (IL-1Ra, e.g., KINERET™ (anakinra; Amgen)), jun, kallikrein 6 (“KLK6”), KGF, ki-ras, kit ligand, stem cell factor (“SCF”), klotho (“KL”), L-myc, large tumor suppressor (“LATS1”), LDL receptor (“LDLR”), leptin (“LEP”), leptin receptor (“LEPR”), leucine amino peptidase-3 (“LAP3”), leukemia inhibitory factor (“LIF”), leukemia inhibitory factor receptor (“LIFR”), livin, luteinizing hormone, luteinizing hormone releasing hormone, macrophage migration inhibitory factor (“MIF”), major histocompatibility complex class I chain-related gene A (“MICA”), major histocompatibility complex class I chain-related gene B (“MICB”), matrix metalloproteinase 9 (“MMP9”), matrix metalloproteinase 12 (“MMP12”), max interacting protein 1 (“MXI1”), MCC, MDM2, METH-1, METH-2, methyl-CpG-binding endonuclease (“MBD4”), monoamine oxidase-A (“MAOA”), monoamine oxidase-B (“MAOB”), monocyte chemotactic protein 1 (“MCPI”), mos, MTS1, myc, myotrophin, N-acetyltransferase, N-cadherin, N-methyl D-aspartate (“NMDA”) receptor, NAD(P)-dependent steroid dehydrogenase (“NSDHL”), natural resistance-associated macrophage protein (“NRAM P”), neural cell adhesion molecule 1 (“NCAM1”), neuron growth associated protein 43 (“GAP-43”), NF1, NF2, nm23, nuclear factor of kappa light polypeptide gene enhancer in B-cells 1 (“NFKB 1”), OSM, osteopontin (“OPN”), P-glycoprotein-1 (“PGYI”), p38 MAP kinase (“p38” or “MAPK14”), p53, p300/CBP associated factor(“PCAF”), parathyroid hormone, peroxin-1 (“PEX1”), peroxisome assembly factor-2 (“PEX6”), peroxisome proliferator-activated receptor-gamma (“PPARg”), phenylalanine hydroxylase, phosphodiesterase, phosphotyrosyl-protein phosphatase (“PTP-1B”), placental growth factor (“PGF”), plasminogen activator inhibitor protein (“PAI1”), pleiotrophin (“PTN”), poly(rc) binding protein 2 (“PCBP2”), progranulin (“PCDGF” or “GRN”), prolactin (“PRL”), proliferating cell nuclear antigen (“PCNA”), protein kinase B/Akt (“AKT1”), protein kinase C gamma (“PKCg”), protein-tyrosine phosphatase, 4A, 3 (“PTP4A3”), psoriasin (“PSOR1”), ras, resistin, retinoblastoma (“Rb”), retinoblastoma 1 (“Rb1”), retinoblastoma-binding protein 1-like 1 (“RBBP1L1”), ribonuclease/angiogenin inhibitor (“RNH”), S100 calcium-binding protein A8 (“MRP8”), signal transducer and activator of transcription (“STAT”)-1, STAT-2, STAT-3, STAT-4, STAT-5, STAT-6, soluble-type polypeptide FZD4S (“FZD4S”), somatotropin, src, survivin, T-cell lymphoma invasion and metastasis 1 (“TIAM1”), TEK tyrosine kinase (“TIE2”), telomerase, thrombomodulin (“THBD” or “THRM”), thrombopoietin (“THPO” or “TPO”), human triosephosphate isomerase (“TPI1”), thyroid hormone, thyroid stimulating hormone, tissue factor, tissue inhibitor of metalloprotease 1 (“TIMP1”), tissue inhibitor of metalloprotease 2 (“TIMP2”), tissue inhibitor of metalloprotease 4 (“TIMP4”), uncoupling protein 2 (“UCP2”), urokinase plasminogen activator (“uPA”), utrophin (“UTRN”), v-myc myelocytomatosis viral oncogene homolog, vanilloid receptor subunit 1 (“VR1”), EphA2, virion infectivity factor (“VIF”), VLA-4, HIV gp120, HIV nef, RSV F glycoprotein, RSV G glycoprotein, influenza virus neuraminidase, influenza virus hemagglutinin, HTLV tax, herpes simplex virus glycoprotein (e.g., gB, gC, gD, and gE), and hepatitis B surface antigen.

### 4.4 CHARACTERIZATION OF A PROTEIN OF INTEREST

In a certain embodiment, the methods and compositions of the invention provide not only high levels of an expressed protein of interest, but a protein of interest which is stable, pure and/or biologically active or functional. In this respect, the invention provides methods of characterizing a protein of interest which is expressed by the methods of the invention, which generally involve monitoring the integrity, stability and/or purity of an expressed protein of interest, particularly antibodies. For example, in certain embodiments of the invention, SDS-PAGE can be used to assess purity; size exclusion high performance liquid chromatography can be used to test for integrity and aggregation; activity or biological assays can be used to determine efficacy and/or potency; ultraviolet absorbance can be used to assess concentration; and isotyping assays can be used for identification. Further, enzyme-linked immunoabsorbant assay (ELISA), Western-blotting and DNA hybridization or polymerase chain reaction (PCR) can be used to assay for process contaminants. Finally, the invention encompasses methods of characterizing a protein by determining its primary, secondary, or tertiary structure; its carbohydrate content; its charge isoforms; or its hydrophobic interactions.

There are various methods available for assessing the stability of protein formulations, including antibody formulations, based on the physical and chemical structures of the proteins as well as on their biological activities. For example, to study denaturation of proteins, methods such as charge-transfer absorption, thermal analysis, fluorescence spectroscopy, circular dichroism, NMR, and high performance size exclusion chromatography (HPSEC), are available. See, for example, Wang et al., 1988, J. of Parenteral Science & Technology 42(Suppl):S4-S26.

Reduced capillary gel electrophoresis (RCGE) and HPSEC are the most common and simplest methods to assess the formation of protein aggregates, protein degradation, and protein fragmentation. Accordingly, the stability of the liquid formulations of the present invention may be assessed by these methods.

For example, the stability of a protein produced by the present invention or contained in the compositions of the invention may be evaluated by HPSEC or rCGE, wherein the percent area of the peaks represents the non-degraded protein. For example, approximately 250 μg of a protein (approximately 25 μl of a liquid formulation comprising 10 mg/ml of said antibody or antibody fragment) is injected onto a TosoH Biosep TSK G3000SW_{XL} column (7.8 mm×30 cm) fitted with a TSK SW XL guard column (6.0 mm×4.0 cm). The protein is eluted isocratically with 0.1 M sodium phosphate dibasic containing 0.1 M sodium sulfate and 0.05% sodium azide, at a flow rate of 0.8 to 1.0 ml/hour. Eluted protein is detected using UV absorbance at 280 nm. A reference standard is run in the assay as a control, and the results are reported as the area percent of the product monomer peak compared to all other peaks excluding the included volume peak observed at approximately 12 to 14 minutes. Peaks eluting earlier than the monomer peak are recorded as percent aggregate.

In one embodiment, the proteins produced by the present invention or contained in compositions of the invention exhibit low to undetectable levels of aggregation as measured by HPSEC or rCGE, that is, no more than 5%, no more than 4%, no more than 3%, no more than 2%, no more than 1%, and most preferably no more than 0.5% aggregate by weight protein, and low to undetectable levels of fragmentation, that is, 80% or higher, 85% or higher, 90% or higher, 95% or higher, 98% or higher, or 99% or higher, or 99.5% or higher of the total peak area in the peak(s) representing intact protein. In the case of SDS-PAGE, the density or the radioactivity of each band stained or labeled with radioisotope can be measured and the % density or % radioactivity of the band representing non-degraded protein can be obtained.

The stability of the proteins produced by the present invention or contained in the compositions of the invention can be also assessed by any assay which measures the biological activity of the protein. The biological activities of antibodies, e.g., include but are not limited to, antigen-binding activity, complement-activation activity, Fc-receptor binding activity, and so forth. Antigen-binding activity of antibodies can be measured by any method known to those skilled in the art, including but not limited to ELISA, radioimmunoassay, Western blot, and the like. Complement-activation activity can be measured by a C3a/C4a assay in the system where the antibody which immunospecifically binds to an epitope in the presence of the complement components with the cells expressing the epitope. Also see Harlow et al., Antibodies: A Laboratory Manual, (Cold Spring Harbor Laboratory Press, 2nd ed. 1988), incorporated by reference herein in its entirety. An ELISA based assay, e.g., may be used to compare the ability of an antibody or fragment thereof to immunospecifically bind to a reference standard.

The purity of the proteins produced by the invention or contained in the compositions of the invention may be measured by any method well-known to one of skill in the art such as, e.g., HPSEC. The sterility of an antibody, e.g., may be assessed as follows: sterile soybean-casein digest medium and fluid thioglycollate medium are inoculated with a test liquid antibody formulation by filtering the liquid antibody formulation through a sterile filter having a nominal porosity of 0.45 μm. When using the Sterisure™ or Steritest™ method (Millipore, Billerica, Mass.), each filter device is aseptically filled with approximately 100 ml of sterile soybean-casein digest medium or fluid thioglycollate medium. When using the conventional method, the challenged filter is aseptically transferred to 100 ml of sterile soybean-casein digest medium or fluid thioglycollate medium. The media are incubated at appropriate temperatures and observed three times over a 14 day period for evidence of bacterial or fungal growth.

In a particular embodiment, the stability, purity and/or integrity of a protein is assessed periodically, e.g., once every 24 hours, during the fed-batch process of the invention. In another embodiment, the stability, purity and/or integrity of a protein of interest is assessed each time the process is scaled-up.

### 4.4.1 Assays for Antigenic Peptides

The present invention provide Listeria-based vaccines comprising Listeria bacteria engineered to express an antigenic peptide. Any assay known in the art for determining whether a peptide is a T cell epitope or a B cell epitope may be employed in testing antigenic peptides for suitability in the present methods and compositions.

For example, ELISPOT assays and methods for intracellular cytokine staining can be used for enumeration and characterization of antigen-specific CD4+and CD8⁺ T cells. Lalvani et al. (1997) J. Exp. Med. 186:859-865; Waldrop et al. (1997) J. Clin Invest. 99:1739-1750.

Antigenic peptides can be determined by screening synthetic peptides corresponding to portions of the antigen. Candidate antigenic peptides can be identified on the basis of their sequence or predicted structure. A number of algorithms are available for this purpose.

Exemplary protocols for such assays are presented below.

### 4.4.1.1 Peptides that Display Immunogenicity of the Antigen

The ability of antigenic peptides to elicit a specific antibody responses in mammals can be examined, for example, by immunizing animals (e.g., mice, guinea pigs or rabbits) with individual antigen peptides emulsified in Freund's adjuvant.

After three injections (5 to 100 μg peptide per injection), IgG antibody responses are tested by peptide-specific ELISAs and immunoblotting against the antigen.

Antigenic peptides which produce antisera that react specifically with the antigenic peptides of interest and also recognized full length antigenic protein in immunoblots are said to display the antigenicity of the antigenic peptide of interest.

### 4.4.1.2 CD4⁺ T-Cell Proliferation Assay

For example, such assays include in vitro cell culture assays in which peripheral blood mononuclear cells (“PBMCs”) are obtained from fresh blood of a patient with a disease involving overexpression of an antigenic peptide, such as EphA2, and purified by centrifugation using FICOLL-PLAQUE PLUS (Pharmacia, Upsalla, Sweden) essentially as described by Kruse and Sebald, 1992, EMBO J. 11:3237-3244. The peripheral blood mononuclear cells are indubated for 7-10 days with candidate EphA2 antigenic peptides. Antigen presenting cells may optionally be added to the culture 24 to 48 hours prior to the assay, in order to process and present the antigen. The cells are then harvested by centrifugation, and washed in RPMI 1640 media (GibcoBRL, Gaithersburg, Md.). 5×10⁴ activated T cells/well are in RPMI 1640 media containing 10% fetal bovine serum, 10 mM HEPES, ph 7.5, 2 mM L-glutamine, 100 units/ml penicillin G, and 100 μg/ml streptomycin sulphate in 96 well plates for 72 hrs at 37° C., pulsed with 1 pCi ³H-thymidine (DuPont NEN, Boston, Mass.)/well for 6 hrs, harvested, and radioactivity measured in a TOPCOUNT scintillation counter (Packard Instrument Col., Meriden, Conn.).

4.4.1.3 Intracellular Cytokine Staining (ICS)

Measurement of antigen-specific, intracellular cytokine responses of T cells can be performed essentially as described by Waldrop et aL, 1997, J. Clin. Invest. 99:1739-1750; Openshaw et al., 1995, J. Exp. Med. 182:1357-1367; or Estcourt et aL, 1997, Clin. Immunol. Immunopathol. 83:60-67. Purified PBMCs from patients with a disease involving, e.g., EphA2-overexpressing cells are placed in 12×75 millimeter polystyrene tissue culture tubes (Becton Dickinson, Lincoln Park, N.J.) at a concentration of 1×10⁶ cells per tube. A solution comprising 0.5 milliliters of HL-1 serum free medium, 100 units per milliliter of penicillin, 100 units per milliliter streptomycin, 2 millimolar L glutamine (Gibco BRL), varying amounts of individual EphA2 antigenic candidate peptides, and 1 unit of anti-CD28 mAb (Becton-Dickinson, Lincoln Park, N.J.) is added to each tube. Anti-CD3 mAb is added to a duplicate set of normal PBMC cultures as positive control. Culture tubes are incubated for 1 hour. Brefeldin A is added to individual tubes at a concentration of 1 microgram per milliliter, and the tubes are incubated for an additional 17 hours.

PBMCs stimulated as described above are harvested by washing the cells twice with a solution comprising Dulbecco's phosphate-buffered saline (dPBS) and 10 units of Brefeldin A. These washed cells are fixed by incubation for 10 minutes in a solution comprising 0.5 milliliters of 4% paraformaldehyde and dPBS. The cells are washed with a solution comprising dPBS and 2% fetal calf serum (FCS). The cells are then either used immediately for intracellular cytokine and surface marker staining or are frozen for no more than three days in freezing medium, as described (Waldrop et al., 1997, J. Clin. Invest. 99:1739-1750).

The cell preparations were rapidly thawed in a 37° C. water bath and washed once with dPBS. Cells, either fresh or frozen, are resuspended in 0.5 milliliters of permeabilizing solution (Becton Dickinson Immunocytometry systems, San Jose, Calif.) and incubated for 10 minutes at room temperature with protection from light. Permeabilized cells are washed twice with dPBS and incubated with directly conjugated mAbs for 20 minutes at room temperature with protection from light. Optimal concentrations of antibodies are predetermined according to standard methods. After staining, the cells were washed, refixed by incubation in a solution comprising dPBS 1% paraformaldehyde, and stored away from light at 4° C. for flow cytometry analysis.

### 4.4.1.4 ELISPOT Assays

The ELISPOT assay measures Th1-cytokine specific induction in murine splenocytes following Listeria vaccination. ELISPOT assays are performed to determine the frequency of T lymphocytes in response to endogenous antigenic peptide stimulation, and are as described in Geginat, et al., 2001, J. Immunol. 166:1877-1884. Balb/c mice (3 per group) are vaccinated with L. monocytogenes expressing candidate antigenic peptides or HBSS as control. Whole mouse spleens are harvested and pooled five days after vaccination. Single cell suspensions of murine splenocytes are plated in the presence of various antigens overnight in a 37° C. incubator.

Assays are performed in nitrocellulose-backed 96-well microtiter plates coated with rat anti-mouse IFN-γ mAb. For the testing of the candidate antigenic peptide, a 1×10⁻⁵ M peptide solution is prepared. In round-bottom 96-well microtiter plates per well 6×10⁵ unseparated splenocytes in 135 μl culture medium (a modification of Eagle's medium (Life Technologies, Eggenstein, Germany) supplemented with 10% FCS, 100 U/ml penicillin, 100 μg/ml streptomycin, 1×10⁻⁵ M 2-ME, and 2 mM glutamine) are mixed with 15 μl of the 1×10⁻⁵ M peptide solution to yield a final peptide concentration of 1×10⁻⁶ M. After 6 h of incubation at 37° C., cells are resuspended by vigorous pipetting, and 100 μl or 10 μl of cell suspension (4×10⁵/well or 4×10⁴/well, respectively) is transferred to Ab-coated ELISPOT plates and incubated overnight at 37° C. In the ELISPOT plates, the fmal volume was adjusted to 150 μl to ensure homogenous distribution of cells.

Purified CD4⁺ or CD8⁺ T cells are tested in a modified assay as follows: 15 μl prediluted peptide (1×10⁻⁵ M) is directly added to Ab-coated ELISPOT plates and mixed with 4×10⁵ splenocytes from nonimmune animals as APC to yield a final volume of 100 μl. After 4 h of preincubation of APC at 37° C., 1×10⁵ CD4⁺ or CD8⁺ cells purified from L. monocytogenes-immune mice are added per well in a volume of 50 μl and plates are incubated overnight at 37° C. The ELISPOT-based ex vivo MHC restriction analysis is performed after loading of cell lines expressing specific MHC class I molecules with 1×10⁻⁶ M peptide for 2 h at 37° C. Subsequently, unbound peptides are washed off (four times) to prevent binding of peptides to responder splenocytes. Per well of the ELISPOT plate, 1×10⁵ peptide-loaded APC are mixed with 4×10⁵ or 4¹⁰⁴ responder splenocytes in a final volume of 150 μl. After overnight incubation at 37° C., ELISPOT plates are developed with biotin-labeled rat anti-mouse IFN-γ mAb, HRP streptavidin conjugate, and aminoethylcarbazole dye of spots per splenocytes seeded. The specificity and sensitivity of the ELISPOT assay is controlled with IFN-γ secreting CD8 T cell lines specific for a control antigen.

### 4.5 RECOVERY AND PURIFICATION OF EXPRESSED PROTEINS

The expressed proteins generated by the methods of the invention can be recovered and purified using any method known in the art. The following section, by way of illustration and not limitation, provides methods of recovery and purification of a protein of interest.

Therapeutic proteins should be prepared so that the risk of containing harmful contaminating agents, such as viruses, pyrogens, DNA fragments and immunogenic proteins, is very low. At the same time it is desirable to maintain the activity and specificity of the protein.

### 4.5.1 Recovery

A protein of interest produced by the methods of the invention can be recovered by using any technique known in the art. Centrifugation and filtration are often used for clarification of harvested cell culture media. Continuous flow centrifuges are useful for large volumes; using this method, solids are collected in a container which can be periodically emptied. For large-scale commercial or industrial culture processes where hundreds or thousands of liters of the harvested media are produced, intermittent discharge centrifuges are available that collect solids in a container and periodically ejects the contents so that operation does not need to be stopped to empty the container.

Alternatively, clarification by filtration is available using conventional dead-end filters or cross-flow filters. In cross-flow filtration, the media are continuously recirculated through a filtration membrane, which has the advantage that solids (cells and cell debris) can be recovered and the membrane is reusable. In dead-end filtration, the solids cannot be extricated from the filter, and thus the filter must be replaced with every use. Moreover, several dead-end filters are often placed in series with decreasing pore size, with the first filter retaining solids, while the last generally sterilizing the fluid.

Alternatively, hollow fiber technology, including, but not limited to, the use of hollow fiber cartridges (Amersham Biosciences) and hollow fiber membranes can be used during harvesting.

### 4.5.2 Purification

The proteins produced by the methods of the invention may be purified using any technique known in the art, including, by way of example and not limitation, chromatography (e.g., ion exchange, affinity and sizing column chromatography), centrifugation, differential solubility and diafiltration. Examples of such techniques are presented in detail in Harlow and Lane, 1988, Antibodies: A Laboratory Manual, Cold Harbor Spring Press, New York and Strategies for Protein Purification and Characterization; A Laboratory Manual, Eds: Marshak et al., Cold Spring Harbor Press, New York, 1996, the disclosures of which are incorporated by reference herein in their entirety.

### 4.6 USES OF LISTERIA

### 4.6.1 Vaccine Compositions

Recombinant forms of Listeria are useful as vaccines. A recombinant form of Listeria can express a tumor associated antigen, a viral protein, or a fusion protein comprising a tumor associated antigen or viral protein and a Listeria protein such as listeriolysin. The use of a tumor associated antigen in this form can induce an immune response to the tumor. Such use is described in U.S. Pat. No. 6,565,852, herein incorporated by reference in its entirety.

Most cancers are associated with more than one antigen. Examples of tumors that express more than one tumor antigen include, but are not limited to, breast cancer which has been shown to be associated with MUC-1, HER-2/neu, MAGE, p53, T/Tn and CEA, colon cancer which has been shown to be associated with MUC-2 and MUC-4, CEA, p53 and the MAGE family, melanoma which has been shown to be associated with members of the MAGE family, MART-1 and gp100, and prostate cancer which has been associated with GM2, Tn, sTn, Thompson-Friedenreich antigen (TF), MUC1, MUC2, the beta chain of human chorionic gonadotropin (hCG beta), HER2/neu, PSMA and PSA. In fact, panels of antigens have been suggested for use in immunotherapy against cancer to compensate for the fact that antigen-loss variants of the tumors can grow out under immune system pressure (Zhang et al. Clin. Cancer Res. 1998 4:2669; Kawashima et al. Hum. Immunol. 1998 59:1). Accordingly, a vaccine may comprise a cocktail of recombinant L. monocytogenes, each expressing a different tumor associated antigen or a cocktail of fusion proteins, each fusion protein comprising a different tumor associated antigen fused to a truncated form of listeriolysin.

For preparation of a vaccine containing live Listeria, it is preferable to use an attenuated mutant strain. In a preferred embodiment of the present invention, the attenuated bacterium is a mutant of wild-type Listeria which invades host cells and is released into the cytosol of the infected cells with similar efficiencies as the wild-type strain, but is impaired in intra- and intercellular movement. Mutant bacteria are therefore unable to move from one infected cell into a neighboring cell (cell-to-cell spread). This illustrates a decreased ability (e.g., as compared to wild type strains) in intra- and inter-cellular movement.

In a preferred embodiment, the invention provides Listeria bacteria engineered to express an antigenic peptide, e.g., EphA2, and the use of such Listeria to manage, treat or prevent diseases associated with overexpression of an antigenic peptide, e.g., EphA2.

A Listeria-based EphA2 vaccine may comprise one or more strains of Listeria that express an EphA2 antigenic peptide. In other embodiments, a Listeria-based EphA2 vaccine may comprise a Listeria strain that has been engineered to express one or more EphA2 antigenic peptides.

In a preferred embodiment, the Listeria-based EphA2 vaccine of the invention comprises the species Listeria monocytogenes.

### 4.6.2 Therapeutic and Prophylactic Applications

A vaccine expressed using the methods of the invention may have therapeutic activity for the treatment or prevention of a disease or disorders. Diseases and disorders which can be treated or prevented with such a vaccine, include, but are by no means not limited to, cancer, autoimmune diseases and disorders, infectious diseases and disorders, and diseases involving aberrant angiogenesis. Representative hyperproliferative diseases and disorders include, but are not limited to, cancer; mucin-related disorders, such as asthma, chronic obstructive pulmonary disease (COPD) chronic bronchitis, bronchietactis, and cystic fibrosis; restenosis; and neointimal hyperplasia. Representative diseases involving aberrant angiogenesis include but are not limited to macular degeneration, diabetic retinopathy, retinopathy of prematurity, vascular restenosis, infantile hemangioma, verruca vulgaris, psoriasis, Kaposi's sarcoma, neurofibromatosis, recessive dystrophic epidermolysis bullosa, rheumatoid arthritis, ankylosing spondylitis, systemic lupus, psoriatic arthropathy, Reiter's syndrome, and Sjogren's syndrome, endometriosis, preeclampsia, atherosclerosis and coronary artery disease.

In certain embodiments, the hyperproliferative disease is cancer. In certain embodiments, the cancer is of an epithelial cell origin and/or involves cells that overexpress EphA2 relative to non-cancer cells having the tissue type of said cancer cells. In specific embodiments, the cancer is a cancer of the skin, lung, colon, breast, ovary, esophageal, prostate, bladder or pancreas or is a renal cell carcinoma or melanoma. In yet other embodiments, the cancer is of a T cell origin. In specific embodiments, the cancer is a leukemia or a lymphoma. In yet other embodiments, the hyperproliferative disorder is non-neoplastic. In specific embodiments, the non-neoplastic hyperproliferative disorder is an epithelial cell disorder. Exemplary non-neoplastic hyperproliferative disorders are asthma, chronic pulmonary obstructive disease, lung fibrosis, bronchial hyper responsiveness, psoriasis, and seborrheic dermatitis.

The invention, by way of example and not limitation, encompasses treatment of mammals, including, by way of example and not limitation, humans, household pets and farm animals; and treatment of reptiles, birds and fish.

The invention also contemplates combination therapy wherein a protein of interest produced using the methods of the invention is administered concurrently with, before or after, another therapeutic agent known in the art, for the treatment or prevention of a disease or disorder.

### 4.6.3 Protein Production

The methods of the invention also encompass the production of proteins from Listeria cells. Since Listeria are gram positive, any protein that is secreted will end up in the growth medium where it can be easily harvested using standard methods.

### 4.7 PHARMACEUTICAL FORMULATIONS

In one embodiment, any of the proteins produced using the methods of the invention can be incorporated into pharmaceutical compositions. Pharmaceutical compositions for use in accordance with the present invention may be formulated in conventional manner using one or more physiologically acceptable carriers or excipients.

Thus, the proteins of the invention may be formulated for administration by inhalation or insufflation (either through the mouth or the nose) or oral, parenteral or mucosal (such as buccal, vaginal, rectal, sublingual) administration. In a particular embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the proteins are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Proteins may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Proteins may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, proteins may be formulated with suitable polymeric or hydrophobic materials (for example as an emulsion in an acceptable oil) or ion exchange resins, or as sparingly soluble derivatives, for example, as a sparingly soluble salt.

The invention also provides that the protein formulation is packaged in a hermetically sealed container such as an ampoule or sachette indicating the quantity. In one embodiment, the protein formulation is supplied as a dry sterilized lyophilized powder or water-free concentrate in a hermetically sealed container and can be reconstituted, e.g., with water or saline to the appropriate concentration for administration to a subject.

In certain embodiments of the invention, a protein is formulated at 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 ml/mL, 225 mg/mL, 250 mg/mL, 275 mg/mL and 300 mg/mL for intravenous injections and at 5 mg/mL, 10 mg/mL, 25 mg/mL, 50 mg/mL, 75 mg/mL, 100 mg/mL, 125 mg/mL, 150 mg/mL, 175 mg/mL, 200 ml/mL, 225 mg/mL, 250 mg/mL, 275 mg/niL and 300 mg/mL for intravenous injections or repeated subcutaneous administration.

In certain embodiments, the antigenic peptide-expressing Listeria of the invention are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection. In other embodiments, the antigenic peptide-expressing Listeria of the invention are formulated at amounts ranging between approximately 1×10² CFU/ml to approximately 1×10¹² CFU/ml, for example at 1×10² CFU/ml, 5×10² CFU/ml, 1×10³ CFU/ml, 5×10³ CFU/ml, 1×10⁴ CFU/ml, 5×10⁴ CFU/ml, 1×10⁵ CFU/ml, 5×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 5×10¹⁰ CFU/ml, 1×10¹¹ CFU/ml, 5×10¹¹ CFU/ml, or 1×10¹² CFU/ml.

The compositions may, if desired, be presented in a pack or dispenser device that may contain one or more unit dosage forms containing the active ingredient. The pack may, for example, comprise metal or plastic foil, such as a blister pack. The pack or dispenser device may be accompanied by instructions for administration.

In certain preferred embodiments, the pack or dispenser contains one or more unit dosage forms and no more than the recommended dosage formulation as determined in the Physician's Desk Reference (56^{th} ed. 2002, herein incorporated by reference in its entirety) for a particular disease or disorder therapy.

### 4.7.1 Dosages

The amount of the composition of the invention which will be effective in the treatment, prevention, management or amelioration of a disease or a disorder or one or more symptoms thereof can be determined by standard research techniques. For example, the dosage of the composition which will be effective in the treatment, prevention, management, or amelioration of cancer or one or more symptoms thereof can be determined by administering the composition to an animal model such as, e.g., animal models known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the cancer, or other disease or disorder, and should be decided according to the judgment of the practitioner and each patient's circumstances. Effective doses may be extrapolated from dose-response curves derived from in vitro or animal model test systems.

For peptides, polypeptides, proteins, fusion proteins, and antibodies, the dosage administered to a patient is typically 0.01 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

In a preferred embodiment, the dose of an antibody or antibody fragment is generally 0.1 to 10 mg/kg/week, preferably 1 to 9 mg/kg/week, more preferably 2 to 8 mg/week, even more preferably 3 to 7 mg/kg/week, and most preferably 4 to 6 mg/kg/week. In another embodiment, a subject, preferably a human, is administered one or more doses of a prophylactically or therapeutically effective amount of an antibody or antibody fragment wherein the dose of a prophylactically or therapeutically effective amount of the antibody or antibody fragment in the liquid formulation administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject, preferably a human, is administered one or more doses of a prophylactically or therapeutically effective amount of an antibody or antibody fragment, wherein the dose of a prophylactically or therapeutically effective amount of the antibody or antibody fragment in the liquid formulation of the invention administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

Exemplary doses of a small molecule (peptide or polypeptide) include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

With respect to the dosage of Listeria in the Listeria-based vaccines of the invention, the dosage is based on the amount colony forming units (c.f.u.). Generally, in various embodiments, the dosage ranges are from about 1.0 c.f.u./kg to abojut 1×10¹⁰ c.f.u./kg; from about 1.0 c.f.u./kg to about 1×10⁸ c.f.u./kg; from about 1×10² c.f.u./kg to about 1×10⁸ c.f.u./kg; and from about 1×10⁴ c.f.u./kg to about 1×10⁸ c.f.u./kg. Effective doses may be extrapolated from dose-response curves derived animal model test systems. In certain exemplary embodiments, the dosage ranges are 0.001-fold to 10,000-fold of the murine LD₅₀, 0.01-fold to 1,000-fold of the murine LD₅₀, 0.1-fold to 500-fold of the murine LD₅₀, 0.5-fold to 250-fold of the murine LD₅₀, 1-fold to 100-fold of the murine LD₅₀, and 5-fold to 50-fold of the murine LD₅₀. In certain specific embodiments, the dosage ranges are 0.00.1-fold, 0.01-fold, 0.1-fold, 0.5-fold, 1-fold, 5-fold, 10-fold, 50-fold, 100-fold, 200fold, 500-fold, 1,000-fold, 5,000-fold or 10,000-fold of the murine LD₅₀.

The dosages of prophylactic or therapeutically agents are described in the Physicians' Desk Reference (56^{th} ed., 2002).

### 4.8 KITS

The invention provides a pack or kit comprising one or more containers filled with a Listeria-based vaccine of the invention or a component of a Listeria-based vaccine of the invention. Additionally, one or more other prophylactic or therapeutic agents useful for the treatment of a cancer or other hyperproliferative disorder can also be included in the pack or kit. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5. EXAMPLES

### 5.1 General Methods

Example runs were performed as follows, except where otherwise noted.

Preparation of the Equipment

The 7L autoclaveable bioreactor vessel (Applikon) was connected with all peripheral equipment including probes, feed bottles, and other equipment with all ports sealed to form a closed, but vented system. Included in the bioreactor was 0.5 mL per L batch volume of culture of Antifoam 204, Sigma Catalog# A6426. The vessel was autoclaved for 30 minutes at greater than 121° C. When the cycle was completed the sterile vessel was cooled to room temperature. The vessel was then charged with 4 L of the batch growth medium, through a 0.22 μm filter. The reactor, with 3 rushton-type impellors, was agitated at 1000 RPM. Air was sparged into the reactor at a flowrate of 4 L/min. The reactor was heated to 37° C. When the temperature reached steady-state, the dissolved oxygen probes were calibrated at 100% air saturation.

TABLE 1
Inoculum Expansion Medium

| Component | Concentration | |
|---|---|---|
| Yeastolate, ultrafiltered | 25 | g/L |
| Dextrose, anhydrous (glucose) | 10 | g/L |
| KH₂PO₄ | 9 | g/L |
| 10 N NaOH | 5 | mL/L |

Vial Thaw and Inoculum Expansion

A 1 mL vial of Listeria was thawed from −80° C. into 50-100 mLs of inoculum expansion medium in a 500 mL vented-cap shake flask. This culture was expanded overnight in a shaker/incubator at 37° C. and 200 RPM. After reaching a concentration of 3-5 OD units, the inoculum was introduced into the bioreactor using a sterile syringe at a volume of 0.5% of the batch culture.

Bioreactor Batch Phase

The basal media in the bioreactor was the same yeast-extract based medium used for the inoculum. (25 g/L yeast extract, 9 g/L KH₂PO₄, 10 g/L glucose) The growth of the Listeria culture was analyzed for cell growth using periodic optical density measurements. The culture was also periodically plated for measurement of the viable cell concentration in units of CFU/mL. The culture was grown in the batch phase until the carbon source required for growth was exhausted or nearly exhausted. The pH of the reactor was controlled at 7.2 using a solution of 3M NH40H. The dissolved oxygen setpoint was 50%, below which oxygen was sparged into the bioreactor. The culture was agitated at a rate of 1000 RPM. The temperature was controlled at 37° C. Glucose and lactose were measured using a YSI 2300 STAT Plus Glucose & Lactate Analyzer (YSI Incorporated, Yellow Springs, Ohio).

Feeding Scheme

When the carbon source in the batch medium was exhausted or nearly exhausted, a continuous feed consisting of glucose, and possibly including other components, was introduced into the bioreactor at an initial rate of 2.8 g glucose/hr. The feed rate was exponentially increased with a doubling time of 10 hours, mu=0.07 (1/hr).

Measurement of Colony Forming Units

The culture of Listeria was diluted in a buffer, including Dulbecco's phosphate-buffered saline. The diluted culture was spread onto trypticase soy agar plates. After an incubation period at room temperature or 37° C., the colonies were counted and multiplied by the dilution factor for the viable cell concentration in units of CFU/mL.

### 5.2 Example 1

The following example demonstrates that the fed-batch method of the invention produces a Listeria culture at an OD₆₀₀ of at least 2.2, using a 7 L bioreactor (Applikon).

A vial of Listeria actA in1B was thawed into 100 ml of Defined Medium (w/10 g/L glucose). This medium contains 8.5 g/L K₂HPO₄, 1.5 g/L NaH₂PO₄, 0.5 g/L NH₄Cl, 0.41 g/L MgSO₄-7H₂O, 0.048 g/L FeCl₃-6H₂O, 0.48 g/L nitriloacetic acid, 1 mg/L riboflavin, 1 mg/L thiamine-HCl, 100 μg/L D-biotin, 1μg/L thioctic acid, 76.8 mg/L L-cysteine (free base), 200 mg/L each of: L-Leucine, L-Isoleucine, L-valine, L-Methionine, L-Arginine, and L-Histidine-HCl and incubated at 37° C. at 120 rpm in a Labline Environ-Shaker.

The next morning, the inoculum OD was measured to be 2.13. 100 ml of the inocolum was used to inoculate the bioreactor. The basal medium in the bioreactor was 4 L of Tryptic Soy Broth (Becton Dickinson Bacto™ Tryptic Soy Broth; Franklin Lakes, N.J.) with 10 g/L glucose. The rotation speed was increased to 500 rpm.

At 95 minutes, the agitator speed was increased to 1000 rpm and pH control was turned on to maintain the pH at 7.2 using 3 M NH₄0H. At 855 minutes, 5 ml of 1000× vitamins containing 100 mg/L biotin, 1 g/L riboflavin, 1 g/L thiamine, and 1 mg/L thioctic acid was added. At this point the dissolved oxygen (dO) began to fall. At 875 minutes, feed was started (glucose 650 g/L). The intial feed rate was 45 g glucose/h and increased with a doubling time of 14 h (mu=0.05 1/h). At approximately 1405 minutes, the feed was disabled. At 1490 minutes, 5 ml of 1000x vitamins were added, but did not revive the culture.

TABLE 2
Results from Example 1

| | Dissolved oxygen | | | Lactic acid |
|---|---|---|---|---|
| Time (min) | (DO; %) | OD₆₀₀ | Glucose (g/L) | (g/L) |
| 0 | 100 | | | |
| 75 | 92.35 | 0.264 | 9.36 | 0.088 |
| 235 | 104.9 | 0.78 | 8.83 | 0.231 |
| 310 | 69.15 | 1.62 | 7.87 | 0.273 |
| 445 | 60.5 | 2.56 | 6.49 | 0.382 |
| 505 | 69.85 | 4.0 | 5.49 | 0.462 |
| 805 | 73.0 | 7.6 | 3.33 | 1.32 |
| 1365 | 70.45 | 10.9 | 63.3 | 2.95 |
| 1635 | 70.55 | 8.5 | 60.9 | 3.67 |

An OD₆₀₀ of up to 10.9 was achieved.

### 5.3 Example 2

This example follows more closely the standard procedure outlined in section 5.1. The feed was programmed for a doubling time of 14 h, and the initial feed rate was adjusted to about 2.0 g glucose/hour, based on an estimate of the amount of glucose required for the cells to double in the given time. The initial inoculum of Listeria was at an OD of 1.1. A feed was started at 690 minutes (650 g/L; 50 ml 1000× vitamins). At 2095 minutes, 50 ml 1000× vitamin was added.

TABLE 3
Results from Example 2

| | Dissolved | | | | Feed/base | Feed |
|---|---|---|---|---|---|---|
| Time | oxygen | | Glucose | Lactose | added | mins/base |
| (min) | (DO; %) | OD₆₀₀ | (g/L) | (g/L) | (mls) | mins |
| 0 | 100 | | | | | |
| 630 | 69.1 | 7.3 | 0.107 | 1.15 | | |
| 780 | 88.85 | 7.1 | 0.094 | 0.827 | | |
| 960 | 75.8 | 8.0 | 0.285 | 0.823 | | |
| 1090 | 107.95 | 7.9 | | | | |
| 1210 | 107.95 | 10.7 | 0.113 | 0.618 | 100/260 | 4.01/1.6 |
| 1445 | 108.0 | 14.0 | 0.124 | 0.830 | 140/340 | 5.3/3.3 |
| 2015 | 108.0 | 14.2 | 0.371 | 2.94 | 220/510 | 8.02/7.4 |
| 2275 | 107.95 | 14.2 | 0.500 | 4.24 | 9.52/10.1 | 270/600 |

An OD₆₀₀ of up to 14.2 was achieved.

### 5.4 Example 3

The protocol outlined in Section 5.1 was repeated except for the following differences. The feed was started at 705 minutes (650 g/L glucose, 50 ml of 1000× vitamins). An additional 50 ml of 1000× vitamin was added at 2205 minutes.

TABLE 4
Results from Example 3

| | Dissolved | | | | Feed/base | Feed |
|---|---|---|---|---|---|---|
| Time | oxygen | | Glucose | Lactose | added | mins/base |
| (min) | (DO; %) | OD₆₀₀ | (g/L) | (g/L) | (mls) | mins |
| 0 | 100 | | | | | |
| 645 | 120.45 | 8.0 | 1.21 | 0.654 | | 0/0 |
| 795 | 126.85 | 8.4 | 1.071 | 0.645 | | 1.5/248.9 |
| 975 | 139.9 | 9.1 | 1.083 | 0.468 | | 3.7/250.7 |
| 1095 | 110.55 | 10.1 | | | | 6.1/252.3 |
| 1195 | 137.7 | 14.8 | 0.101 | 0.534 | 70/300 | 8.1/253.6 |
| 1560 | 114.25 | 16.1 | 0.200 | 0.891 | 120/380 | 17.6/258.8 |
| 2130 | 115.65 | 18.7 | 1.71 | 2.35 | 270/540 | 44.1/267.9 |
| 2385 | 120.05 | 17.7 | 5.70 | 3.31 | 320/620 | 63.4/272.5 |

An OD₆₀₀ of up to 18.7 was achieved.

### 5.5 Examples 4-9

The following examples demonstrate the fed-batch method of the invention while varying the concentrations of yeast extract and glucose in the batch medium and the feed.

The bioreactors were inoculated with Listeria monocytogenes using the methods described above. The batch medium contained yeast extract and glucose in concentrations indicated in Table S in addition to 9 g/L KH₂PO₄ and 5 ml/L 10N NaOH. The feed solutions consisted of glucose and yeast extract concentrations as shown in Table 5.

TABLE 5
Summary of Examples 4-9

| | Batch | Batch | Feed | Feed | Maximum | |
|---|---|---|---|---|---|---|
| | YE | Glucose | YE | Glucose | Cell | Maximum |
| Example | (g/L) | (g/L) | (g/L) | (g/L) | Conc'n | OD |
| 4 | 25 | 5 | 250 | 50 | 1.41E+10 | 11.9 |
| 5 | 25 | 0 | 0 | 650 | 7.10E+09 | 11.9 |
| 6 | 50 | 0 | 0 | 650 | 1.80E+10 | 18.3 |
| 7 | 50 | 0 | 0 | 650 | 2.07E+10 | 26.1 |
| 8 | 100 | 0 | 0 | 650 | 2.57E+10 | 39 |
| 9 | 100 | 5 | 0 | 650 | 2.60E+10 | 39 |

Comparison of Examples 4 and 5 shows that feeding yeast extract didn't improve maximum OD (both had a maximum OD of 11.9), but does improve the maximum viabl cell density from 7.1×10⁹ to 1.41×10¹⁰ colony-forming units/mL. In Examples 6-9, the additional yeast extract was incorporated into the batch medium instead of the feed. These runs showed increased optical cell density and viable cell concentration, up to 2.6×10¹⁰ cfu/mL and 39 OD600. Detailed run data is shown in Tables 6 through 11.

TABLE 6
Results from Example 4

| Time (mins) | Optical Density (600 nm) | CFU/mL (×10⁹) |
|---|---|---|
| 310 | 4.2 | 7.3 |
| 390 | 5.7 | 7.7 |
| 530 | 7.5 | 9.1 |
| 620 | 7.9 | 9.4 |
| 765 | 9.3 | 11.3 |
| 940 | 11.3 | 14.1 |
| 1125 | 11.5 | 13.5 |
| 1820 | 11.9 | 10.2 |

TABLE 7
Results from Example 5

| Time (mins) | Optical Density (600 nm) | CFU/mL (×10⁹) |
|---|---|---|
| 240 | 0.3 | 0.3 |
| 340 | 1.58 | 1.8 |
| 430 | 4.3 | 5.1 |
| 735 | 8.2 | 6.1 |
| 1410 | 11.9 | 7.1 |
| 1665 | 11.7 | 4.6 |

TABLE 8
Results from Example 6

| Time (mins) | Optical Density (600 nm) | CFU/mL (×10⁹) |
|---|---|---|
| 10 | 0.277 | 0.04 |
| 100 | 0.27 | 0.05 |
| 230 | 0.65 | 0.63 |
| 295 | 1.37 | n/a |
| 400 | 4 | 7.2 |
| 580 | 8.4 | 13.27 |
| 1465 | 18.3 | 17.9 |
| 1740 | 21.5 | 16 |
| 1900 | 23.2 | 11 |
| 2250 | 23.1 | 7.43 |
| 2910 | 20.8 | 2.23 |

TABLE 9
Results from Example 7

| Time (mins) | Optical Density (600 nm) | CFU/mL (×10⁹) |
|---|---|---|
| 165 | 0.21 | n/a |
| 265 | 0.67 | 0.7 |
| 380 | 1.99 | 3.1 |
| 465 | 4.5 | 7.3 |
| 1440 | 16.4 | 20.7 |
| 1695 | 19 | 18.3 |
| 1915 | 21.9 | 20.4 |
| 2295 | 26.1 | 17.3 |

TABLE 10
Results from Example 8

| Time (mins) | Optical Density (600 nm) | CFU/mL (×10⁹) |
|---|---|---|
| 165 | 0.3 | 0.0033 |
| 300 | 0.46 | 0.0767 |
| 515 | 2.5 | 2.07 |
| 580 | 4.9 | 23.3 |
| 1455 | 23 | 23.1 |
| 1805 | 39 | 25.7 |
| 3255 | 36 | n/a |

TABLE 11
Results from Example 9

| Time (mins) | Optical Density (600 nm) | CFU/mL (×10⁹) |
|---|---|---|
| 185 | 0.3 | 0.02 |
| 315 | 0.41 | 0.0933 |
| 530 | 3.9 | 3.8 |
| 1480 | 25 | 26 |
| 1830 | 39 | 23.3 |
| 3260 | 38 | n/a |

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Various references are cited herein, the disclosures of which are incorporated by reference in their entirety.

What is claimed is:

1. A method for high cell density growth of Listeria, said method comprising fed-batch culturing Listeria cells in culture medium under conditions sufficient and for a time sufficient to achieve an OD₆₀₀ of greater than 2.2.

2. The method of claim 1, wherein said culturing comprises feeding with an additional carbon source after said Listeria culture reaches stationary phase.

3. The method of claim 1 or 2, wherein an OD₆₀₀ greater than about 8.0 is achieved.

4. The method of claim 1 or 2, wherein an OD₆₀₀ greater than about 15.0 is achieved.

5. The method of claim 1 or 2, wherein an OD₆₀₀ greater than about 25.0 is achieved.

6. The method of claim 1, 2, 3, 4, or 5, wherein said OD₆₀₀ is achieved without concentration of the culture medium.

7. The method of claim 2, wherein the carbon source is glucose, yeast extract or a combination thereof.

8. The method of claim 2, wherein the additional carbon source is added at an exponentially increasing rate.

9. The method of claim 2, wherein one or more additional nutrients are added with the additional carbon source.

10. The method of claim 9, wherein the one or more additional nutrients are vitamins or amino acids.

11. The method of claim 1 or 2, wherein the culture medium is tryptic soy medium or yeast growth medium.

12. The method of claim 1 or 2, wherein the culture medium does not contain a protein extract.

13. The method of claim 1 or 2, wherein the culture medium is chemically defined.

14. The method of claim 1 or 2, wherein the Listeria is attenuated.

15. The method of claim 1 or 2, wherein the Listeria recombinantly express a heterologous peptide.

16. The method of claim 15, wherein the heterologous peptide is a tumor-associated antigen.

17. The method of claim 16, wherein the tumor-associated antigen is EphA2.

18. The method of claim 15, wherein the heterologous peptide is a fusion protein.

19. A method for producing a Listeria-based vaccine, said method comprising a) fed-batch culturing Listeria cells in culture medium under conditions sufficient and for a time sufficient to achieve an OD₆₀₀ of greater than 2.2; and (b) recovering the Listeria-based vaccine from said medium.

20. The method of claim 19, wherein said culturing comprises feeding with an additional carbon source after said Listeria culture reaches stationary phase.

21. The method of claim 19 or 20, wherein an OD₆₀₀ greater than about 8.0 is achieved.

22. The method of claim 19 or 20, wherein an OD₆₀₀ greater than about 15.0 is achieved.

23. The method of claim 19 or 20, wherein an OD₆₀₀ greater than about 25.0 is achieved.

24. The method of claim 19, 20, 21, 22, or 23, wherein said OD₆₀₀ is achieved without concentration of the culture medium.

25. The method of claim 20, wherein the carbon source is glucose, yeast extract or a combination thereof.

26. The method of claim 20, wherein the additional carbon source is added at an exponentially increasing rate.

27. The method of claim 20, wherein one or more additional nutrients are added with the additional carbon source.

28. The method of claim 27, wherein the one or more additional nutrients are vitamins or amino acids.

29. The method of claim 19 or 20, wherein the culture medium is tryptic soy medium or yeast growth medium.

30. The method of claim 19 or 20, wherein the culture medium does not contain a protein extract.

31. The method of claim 19 or 20, wherein the culture medium is chemically defined.

32. The method of claim 19 or 20, wherein the Listeria is attenuated.

33. The method of claim 19 or 20, wherein the Listeria-based vaccine comprises a tumor-associated antigen.

34. The method of claim 33, wherein the tumor-associated antigen is EphA2.

35. The method of claim 19 or 20, wherein the Listeria-based vaccine comprises a fusion protein.

36. A method for increasing the yield of a Listeria-based vaccine, said method comprising a) fed-batch culturing Listeria cells in culture medium under conditions sufficient and for a time sufficient to achieve an OD₆₀₀ of greater than 2.2; and (b) recovering the Listeria-based vaccine from said medium, wherein the yield of said Listeria-based vaccine is at least 2-fold higher compared to that achieved by a batch culture using the same Listeria cells.

37. The method of claim 36, wherein said culturing comprises feeding with an additional carbon source after said Listeria culture reaches stationary phase.

38. The method of claim 36 or 37, wherein the yield is at least 3-fold higher.

39. The method of claim 36 or 37, wherein the yield is at least 5-fold higher.

40. A Listeria culture that (1) has been fed-batch cultured, and (2) has an OD₆₀₀ of greater than 2.2.

41. The Listeria culture of claim 40, having an OD₆₀₀ greater than about 8.0.

42. The Listeria culture of claim 40, having an OD₆₀₀ greater than about 15.0.

43. The Listeria culture of claim 40, having an OD₆₀₀ greater than about 25.0.

44. The Listeria culture of claim 40, 41, 42, or 43, wherein said OD₆₀₀ is achieved without concentration of the culture medium.

45. The Listeria culture of claim 40 or 41, wherein the Listeria is attenuated.

46. The Listeria culture of claim 40 or 41, wherein the Listeria recombinantly express a heterologous peptide.

47. The Listeria culture of claim 46, wherein the heterologous peptide is a tumor-associated antigen.

48. The Listeria culture of claim 47, wherein the tumor-associated antigen is EphA2.

49. The Listeria culture of claim 46, wherein the heterologous peptide is a fusion protein.

50. The Listeria culture of claim 40, which is at least 100 liters.
